SEQUENCE LISTING (12 sequences)

SEQ ID NO: 1 - protein, 5 aa, synthetic construct
SGSYG

SEQ ID NO: 2 - skipped

SEQ ID NO: 3 - protein, 11 aa, synthetic construct
GSADSSTYVGM

SEQ ID NO: 4 - protein, 8 aa, synthetic construct
GFTFSDYG

SEQ ID NO: 5 - protein, 8 aa, synthetic construct
ISAAGSFT

SEQ ID NO: 6 - protein, 14 aa, synthetic construct
ARIPSGYRAGEIYT

SEQ ID NO: 7 - protein, 103 aa, synthetic construct
LTQPSSVSANLGETVKIPCSGSSGSYGWYQQKSPGSAPVTLIYDNDKRPSDIPSRFFGSTSGSTSTLTITGVQAEDEAVYFCGSADSSTYVGMFGAGTTLTVL

SEQ ID NO: 8 - protein, 103 aa, synthetic construct
LTQPPSVSVSPGQTVRITCSGSSGSYGWYQQKSPGQAPVTLIYDNDKRPSGIPERFSGSSSGSTSTLTISGVQAEDEADYYCGSADSSTYVGMFGGGTKLTVL

SEQ ID NO: 9 - protein, 121 aa, synthetic construct
TVTLDESGGGLQTPGGGLSLVCKASGFTFSDYGMGWVRQAPGKGLEFVAGISAAGSFTPYGAAVKGRATISRDNGQSTVRLQLNNLRSEDTGIYYCARIPSGYRAGEIYTWGQGTEVIVSS

SEQ ID NO: 10 - protein, 121 aa, synthetic construct
QVQLVESGGGLVQPGGSLRLSCAASGFTFSDYGMGWVRQAPGKGLEWVAGISAAGSFTPYGAAVKGRFTISRDNSKNTVYLQMNSLRAEDTAVYYCARIPSGYRAGEIYTWGQGTLVTVSS

SEQ ID NO: 11 - protein, 231 aa, synthetic construct
LTQPSSVSANLGETVKIPCSGSSGSYGWYQQKSPGSAPVTLIYDNDKRPSDIPSRFFGSTSGSTSTLTITGVQAEDEAVYFCGSADSSTYVGMFGAGTTLTVLGQSSRSSTVTLDESGGGLQTPGGGLSLVCKASGFTFSDYGMGWVRQAPGKGLEFVAGISAAGSFTPYGAAVKGRATISRDNGQSTVRLQLNNLRSEDTGIYYCARIPSGYRAGEIYTWGQGTEVIVSS

SEQ ID NO: 12 - protein, 231 aa, synthetic construct
LTQPPSVSVSPGQTVRITCSGSSGSYGWYQQKSPGQAPVTLIYDNDKRPSGIPERFSGSSSGSTSTLTISGVQAEDEADYYCGSADSSTYVGMFGGGTKLTVLGQSSRSSQVQLVESGGGLVQPGGSLRLSCAASGFTFSDYGMGWVRQAPGKGLEWVAGISAAGSFTPYGAAVKGRFTISRDNSKNTVYLQMNSLRAEDTAVYYCARIPSGYRAGEIYTWGQGTLVTVSS

(19) United States
(12) Patent Application Publication
(10) Pub. No.: US 2025/0340655 A1
(43) Pub. Date: Nov. 6, 2025

(54) EPHA2-TARGETING ANTIBODIES AND THEIR APPLICATIONS IN CANCER TREATMENT

(71) Applicant: TAIPEI MEDICAL UNIVERSITY, Taipei City (TW)

(72) Inventors: Yu-Ching LEE, Taipei City (TW); Yun YEN, Arcadia, CA (US); Fu-Ling CHANG, Taipei City (TW)

(21) Appl. No.: 18/869,756

(22) PCT Filed: May 26, 2023
(86) PCT No.: PCT/US2023/067573

§ 371 Date: Nov. 26, 2024

Related U.S. Application Data
(60) Provisional application No. 63/365,477, filed on May 27, 2022.

(51) Int. Cl. C07K 16/28; A61K 31/7068; A61K 39/00; A61K 47/68; A61P 35/00; G01N 33/574

(52) U.S. Cl. CPC C07K 16/2866; A61K 31/7068; A61K 47/68031; A61K 47/6849; A61P 35/00; G01N 33/57484; A61K 2039/505; C07K 2317/24; C07K 2317/34; C07K 2317/622; C07K 2317/73; C07K 2317/92; G01N 2333/715

(57) ABSTRACT

The present disclosure relates to anti-EphA2 antibody and cancer detection (or diagnosis) and treatment using the anti-EphA2 antibody. The present invention creates anti-EphA2 antibodies, particularly, a single-chain antibody fragments (scFv) and humanized antibody, which have ability in binding to anti-EphA2 and in inhibiting angiogenesis, migration and cancer cell growth.

### PRIORITY INFORMATION

This application is a 371 National Phase of International Patent Application No. PCT/US23/67573, filed on May 26, 2023, which claims benefit of and priority to U.S. Provisional Patent Application No. 63/365,477, filed on May 27, 2022, the contents of which is incorporated by reference in its entirety.

### SEQUENCE LISTING

The instant application contains a Sequence Listing which has been filed electronically in XML format and is hereby incorporated by reference in its entirety. Said XLM copy, created on 15 Nov. 2023, is named US13457-SEQ.xml and is 11,677 bytes in size.

### FIELD OF THE INVENTION

The invention relates to the field of cancer detection (or diagnosis) and treatment. Particularly, the invention relates to an EphA2-targeting antibody and its applications in cancer detection (or diagnosis) and treatment.

### BACKGROUND OF THE INVENTION

Cancer is the uncontrolled growth of abnormal cells anywhere in a body. The abnormal cells are termed cancer cells, malignant cells, or tumor cells. The interactions between erythropoietin producing hepatocyte receptors and ephrins (Ephs/ephrins) control a wide range of biological functions of which have also been implicated in the pathogenesis of human cancers. Eph type A2 (EphA2), a member of tyrosine kinase, interacts with ephrins (ex: ephrin-A1) to trigger bidirectional signaling between cells. Interaction of EphA2 and ephrin-A1 leads to the inhibition of Ras-MAPK activity, resulting the suppression of tumor growth. Moreover, studies have also demonstrated that EphA2 overexpression can drive ligand-independent signaling and induce tumorigenesis. It is therefore believed EphA2 can induce either a negative or positive effect on tumor growth. During tumorigenesis, regular interactions between EphA2 and ephrin-A1 are disturbed, leading to EphA2 overexpression and progression to cancer. Excessive expression of EphA2 has been identified as a notable tumor target in pancreatic cancer diagnosis and treatment. Its higher gene expression is also associated with poor patient outcome. In recent years, several tyrosine kinase inhibitors (TKIs) against EphA2 signaling have been evaluated for their anti-tumor activities. Nevertheless, many of these TKIs have multiple targets, rendering their specificity against EphA2 causing disadvantages in clinical development.

Therefore, there is also a need to develop a specific binding of the antibody to EphA2.

### SUMMARY OF THE INVENTION

The present disclosure provides an isolated anti-EphA2 antibody or an antigen-binding portion thereof, comprising at least one of a light chain CDR1 (L-CDR1) comprising an amino acid residue of SEQ ID NO: 1, or a variant having amino acid sequence with at least 80% identity to any of SEQ ID NO: 1; a light chain CDR2 (L-CDR2) comprising an amino acid residue of DND, or a variant having amino acid sequence with at least 80% identity to any of DND; and a light chain CDR3 (L-CDR3) comprising an amino acid residue SEQ ID NO: 3, or a variant having amino acid sequence with at least 80% identity to any of SEQ ID NO: 3; and at least one of a heavy chain complementarity determining region 1 (H-CDR1) comprising an amino acid residue of SEQ ID NO: 4, or a variant having amino acid sequence with at least 80% identity to any of SEQ ID NO: 4; a heavy chain CDR2 (H-CDR2) comprising an amino acid residue of SEQ ID NO: 5, or a variant having amino acid sequence with at least 80% identity to any of SEQ ID NO: 5; and a heavy chain CDR3 (H-CDR3) comprising an amino acid residue of SEQ ID NO: 6, or a variant having amino acid sequence with at least 80% identity to any of SEQ ID NO: 6; such that said isolated antibody or antigen-binding portion thereof binds to EphA2.

In some embodiments, the antibody of the present disclosure include a monoclonal antibody, chimeric antibody, humanized antibody and human antibody. In some embodiments, the isolated anti-EphA2 antibody or the antigen-binding portion thereof is a single chain Fv (scFv), IgG, Fab, (Fab)₂, or (scFv′)₂.

In some embodiments, the anti-EphA2 antibody or the antigen-binding portion thereof comprises a light chain comprising an amino acid sequence comprising SEQ ID NO: 7 or 8, or a variant having at least 80% identity to SEQ ID NO: 7 or 8, and a heavy chain comprising an amino acid sequence comprising SEQ ID NO: 9 or 10, or a variant having at least 80% identity to SEQ ID NO: 9 or 10. In some embodiments, the anti-EphA2 antibody or the antigen-binding portion thereof comprising a light chain comprising the amino acid sequence of SEQ ID NO: 7 or 8; and a heavy chain comprising the amino acid sequences of SEQ ID NO: 9 or 10.

In some embodiments, the anti-EphA2 antibody or the antigen-binding portion thereof comprises the amino acid sequence of SEQ ID NO: 11 or 12, or a variant having at least 80% identity to SEQ ID NO: 11 or 12. In some embodiments, the anti-EphA2 antibody or the antigen-binding portion thereof comprising the amino acid sequence of SEQ ID NO: 11 or 12.

In further embodiments, the present disclosure provides an isolated antibody (scFv SD5), comprising a light chain having an amino acid sequence as set forth in the sequence comprising SEQ ID NO: 7 or a variant having at least 80% identity to SEQ ID NO: 7, and a heavy chain having an amino acid sequence as set forth in the sequence comprising SEQ ID NO: 9 or a variant having at least 80% identity to SEQ ID NO: 9. Preferably, the sequence identity as mentioned above is at least 90%, 91%, 92%, 93%, 94%, 95%, 96%, 97%, 98% or 99%.

In a further embodiment, the present disclosure provides a humanized antibody (Humanized scFv hSD5), comprising a light chain having an amino acid sequence as set forth in SEQ ID NO: 8 or a variant having at least 80% identity to SEQ ID NO: 8 and a heavy chain having an amino acid sequence as set forth in SEQ ID NO: 10 or a variant having at least 80% identity to SEQ ID NO: 10.

In a further embodiment, the present disclosure provides an isolated antibody (scFv SD5), comprising an amino acid sequence as set forth in SEQ ID NO: 11 or a variant having at least 80% identity to SEQ ID NO: 11. In a further embodiment, the invention comprises a humanized antibody, comprising an amino acid sequence as set forth in SEQ ID NO: 12 or a variant having at least 80% identity to SEQ ID NO: 12.

The present disclosure also provides an antibody-drug conjugate (ADC), comprising the anti-EphA2 antibody of the present disclosure or the antigen-binding portion thereof and a drug-linker structure comprising an antitumor compound connected to the antibody by a linker.

In some embodiments, the antitumor compound is selected from auristatins such as monomethyl auristatin E (MMAE) and monomethyl auristatin F (MMAF), vincristine, vinblastine, methotrexate, platinum-based antitumor agents (cisplatin and derivatives thereof), doxorubicin, calicheamicin, dolastatin 10, maytansinoids, a pyrrolobenzodiazepine dimer, a camptothecin derivative, duocarmycins, amanitin, daunorubicin, mitomycin C, bleomycin, cyclocytidine, and Taxol and derivatives thereof. In some embodiments, the antitumor compound is MMAE.

The present disclosure provides a pharmaceutical composition comprising the anti-EphA2 antibody or the antigen-binding portion thereof or the ADC of the present disclosure and a pharmaceutically acceptable carrier or excipient.

In some embodiments, the pharmaceutical composition further comprises or is used in combination with one or more additional anticancer agents.

In some embodiments, the one or more additional anticancer agents is Gemcitabine.

The present disclosure also provides a method for treating or preventing a EphA2 associated cancer in a subject, comprising administering an anti-EphA2 antibody or the antigen-binding portion thereof or an ADC of the present disclosure to the subject.

The present disclosure also provides a method for inhibiting EphA2 associated cancer cell growth or cancer metastasis in a subject comprising administering an anti-EphA2 antibody or the antigen-binding portion thereof or an ADC of the present disclosure to the subject.

In some embodiments, the EphA2 associated cancer is selected from bile duct cancer, bladder cancer, brain cancer, breast cancer, cervical cancer, colon cancer, esophageal cancer, gastric cancer, gliomas, liver cancer, lung cancer, melanoma, ovarian cancer, pancreatic cancer, prostate cancer, rectal cancer, renal cancer, stomach cancer, thymus cancer, and vulvar cancer. In some embodiments, the EphA2 associated cancer is selected from bladder cancer, brain cancer, bile duct cancer, colon cancer, gastric cancer, and pancreatic cancer.

In some embodiments, each of the above identified compositions and methods of treatment may additionally include an additional anti-tumor drug and the administration of an additional one or more anti-tumor drug.

The present disclosure further provides a method for detecting or diagnosing a EphA2 associated cancer or an elevated risk of future occurrence of a cancer, or predicting a metastasis or prognosis of a cancer in a subject, or monitoring the progression of a cancer in a subject already diagnosed with a EphA2 associated cancer in a subject, comprising contacting a biological sample from a subject with an anti-EphA2 antibody of the present disclosure, quantifying the binding of EphA2 antigen in the sample to the antibody, and comparing said binding to a reference value representing binding between the anti-EphA2 antibody and the EphA2 antigen determined in samples from control subjects not afflicted with a cancer.

The present disclosure further provides a kit for detecting or diagnosing a EphA2 associated cancer or an elevated risk of future occurrence of a EphA2 associated cancer, or predicting a metastasis or prognosis of a cancer, or monitoring cancer progression in a subject, comprising an anti-EphA2 antibody of the present disclosure.

### BRIEF DESCRIPTION OF THE DRAWING

The subject application file contains at least one drawing executed in color. Copies of this patent or patent application publication with color drawing(s) will be provided by the U.S. Patent Office upon request and payment of the necessary fee. The following drawings form part of the present specification and are included to further demonstrate certain aspects of the present disclosure, the inventions of which can be better understood by reference to one or more of these drawings in combination with the detailed description of specific embodiments presented herein. Exemplary embodiments are illustrated in referenced figures. It is intended that the embodiments and figures disclosed herein are to be considered illustrative rather than restrictive.

FIG. 1 shows the cancer types with high EphA2 gene expression were analyzed using GEPIA database.

FIGS. 2A-2D show the analysis and evaluation of the correlation between the EphA2 expression and pancreatic cancer.

FIGS. 3A-3F show the characterization of anti-EphA2 scFvs isolated using phage display technology.

FIGS. 4A-4B show that the scFvs were used to test the cell growth inhibitory effect at different concentrations on four strains of the pancreatic cancer cell.

FIGS. 5A-5D show the inhibition effect of isolated scFvs on the proliferation and migration of PAAD cells.

FIGS. 6A-6D, show the binding specificity of humanized antibody humanized antibody hSD5 to EphA2 and induced tumor suppressor signaling.

FIGS. 7A-7D show the in vivo tumor growth inhibitory effect of Humanized IgG hSD5 on BxPc-3 xenograft mice.

FIGS. 8A-8D show the in vivo tumor growth inhibitory effect of Humanized IgG hSD5 on Mia PaCa-2 xenograft mice.

FIGS. 9A-9B show the epitope definition of IgG hSD5 recognizes the active site of EphA2.

FIG. 10 shows the growth inhibitory response of different pancreatic cancer cell lines treated with serially diluted MMAE.

FIGS. 11A-11D show the growth inhibitory response of different pancreatic cancer cell lines by administration of serially diluted hSD5-ADC.

FIGS. 12A-12B show the cell cycle changes of pancreatic cancer cells.

FIGS. 13A-13B show the BxPc-3 xenograft mouse model was used to test the inhibitory effect on tumor growth in mice after administration of different concentrations of hSD5-ADC and control group IgG-ADC.

FIGS. 14A-14C show the in vivo tumor growth inhibitory effect of hSD5-ADC on BxPc-3 xenograft mice.

FIGS. 15A-15B show the anti-EphA2 hSD5 could recognize the endogenous EphA2 molecule and inhibit the cell growth on gastric cancer.

FIGS. 16A-16B show the expression of EphA2 and the binding ability of anti-EphA2 hSD5 on GBM.

FIGS. 17A-17C show the anti-EphA2 hSD5 could recognize the endogenous EphA2 molecule and inhibit the cell growth on cholagiocarcinoma and bladder cancer.

FIGS. 18A-18C show the anti-EphA2 hSD5 could recognize the endogenous EphA2 molecule and inhibit the cell growth on colon cancer.

FIGS. 19A-19C show the hSD5-ADC can inhibit tumor growth on HCT116 xenograft mouse model.

### DETAILED DESCRIPTION OF THE INVENTION

The terminology herein is used to describe specific embodiments of the invention, but their usage does not delimit the invention, except as outlined in the claims. It is to be understood that both the foregoing general description and the following detailed description are examples and explanatory only and are not restrictive of the subject matter claimed in this application.

Terms such as “a”, “an” and “the” are not intended to refer to only a singular entity, but include the general class of which a specific example may be used for illustration. In this application, the use of “or” means “and/or” unless stated otherwise.

Furthermore, use of the term “including” as well as other forms, such as “includes,” and “included” is not limiting.

As used herein, the terms “tumor,” “cancer” and “carcinoma” are used interchangeably and refer to all neoplastic cell growth and proliferation, whether malignant or benign, and all pre-cancerous and cancerous cells and tissues.

As used herein, the term “biological sample” refers to a sample obtained from a patient. Biological samples, for example, can be obtained from blood, tissue (e.g. tumor), serum, stool, urine, sputum, cerebrospinal fluid, nipple aspirates and supernatant from cell lysate.

As used herein, the term “diagnostic” means identifying the presence or nature of a pathologic condition and includes identifying subjects who are at risk of developing a cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is to measure the proportion of negatives that are correctly identified as such (e.g., the percentage of the subjects who are not diseased and are correctly identified as not having the condition).

As used herein, the terms “detection,” “detecting” and the like, may be used in the context of detecting biomarkers, or of detecting a cancer (e.g. when positive assay results are obtained). In the latter context, “detecting” and “diagnosing” are considered synonymous.

A “test amount” of a marker refers to an amount of a marker present in a sample being tested.

A “control amount” of a marker can be any amount or a range of amount which is to be compared against a test amount of a marker.

The term “at risk of” is intended to mean at increased risk of, compared to a normal subject, or compared to a control group. Thus, a subject “at risk of” developing a cancer is at increased risk compared to a normal population, and a subject “at risk of” a recurrence of a cancer may be considered at increased risk of having a recurrence as compared to the risk of a recurrence among all treated cancer patients.

As used herein, the term “increased risk” or “elevated risk” mean any statistically significant increase in the probability, e.g., that the subject will develop a cancer, or a recurrence thereof.

As used herein, the term “prognosis” refers to the prediction of the likelihood of cancer-attributable death or progression, including recurrence, metastatic spread, and drug resistance, of a neoplastic disease, such as ovarian cancer. The term “poor prognosis” means that the prospect of survival and recovery of disease is unlikely despite the standard of care for the treatment of the cancer (for example, prostate cancer), that is, surgery, radiation, chemotherapy. Poor prognosis is the category of patients whose survival is less than that of the median survival.

As used herein, the term “metastasis” is defined as the spread of cancer from one part of the body to another. A tumor formed by cells that have spread is called a “metastatic tumor” or a “metastasis.”

As used herein, the term “risk of metastasis” refers to a prognostic indication that the cancer in a particular patient, particularly a human patient, will advance to a metastatic state based on statistical predictors. Actual advance to a metastatic state is not required, and adoption of treatment modalities to try to delay or prevent the realization of such risk is anticipated to occur.

As used herein, the expression “reference value” refers to a laboratory value used as reference for the values/data obtained by means of samples obtained from a subject.

As used herein, “determination of a level”, “determining a level” or “measuring a level” typically refer to calculation of an amount or concentration of a particular substance, or to quantifying an intensity of a signal from a probe that represents the amount or concentration of a particular substance.

As used herein, the term “antibody” is used in the broadest sense and specifically covers, for example, single monoclonal antibodies (including agonist, antagonist, and neutralizing antibodies), antibody compositions with polyepitopic specificity, polyclonal antibodies, single chain anti-antibodies, and fragments of antibodies (see below) as long as they specifically bind a native polypeptide and/or exhibit a biological activity or immunological activity of the present invention. According to one embodiment, the antibody binds to an oligomeric form of a target protein, e.g., a trimeric form. The phrase “functional fragment or analog” of an antibody is a compound having a qualitative biological activity in common with an antibody to which it is being referred. For example, a functional fragment or analog of an antibody of this invention can be one which can specifically bind to EGFR. In one embodiment, the antibody can prevent or substantially reduce the ability of an EGFR to induce cell proliferation.

As used herein, the term “isolated antibody” is one which has been identified and separated and/or recovered from a component of its natural environment. Contaminant components of its natural environment are materials which would interfere with diagnostic or therapeutic uses for the antibody, and can include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms known in the art needed to achieve maximal alignment over the full length of the sequences being compared.

As used herein, the term “Fab” indicates an antigen binding fragment of an Ig (regardless of how prepared) including variable domain and first constant domain.

As used herein, the term “Fv” is the minimum antibody fragment which contains a complete antigen-recognition and -binding site. This fragment consists of a dimer of one heavy-and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

As used herein, the term “single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains, which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

As used herein, the term “complementarity determining region” (CDR) refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

As used herein, the term “humanized antibody” refers to a recombinant protein in which the CDRs from an antibody from one species; e.g., a murine or a chicken antibody, are transferred from the heavy and light variable chains of the antibody from the species into human heavy and light variable domains (framework regions). The constant domains of the antibody molecule are derived from those of a human antibody. In some cases, specific residues of the framework region of the humanized antibody, particularly those that are touching or close to the CDR sequences, may be modified, for example replaced with the corresponding residues from the original murine, rodent, subhuman primate, or other antibody. The humanized antibody may be achieved by various methods including (a) grafting only the non-human CDRs onto human framework and constant regions with or without retention of critical framework residues, or (b) transplanting the entire non-human variable domains, but “cloaking” them with a human-like section by replacement of surface residues. Such methods as are useful in practicing the present invention include that disclosed in Padlan, Mol. Immunol., 31 (3): 169-217 (1994).

As used herein, the term “chimeric antibody” refers to a recombinant protein that contains the variable domains of both the heavy and light antibody chains, including the complementarity determining regions (CDRs) of an antibody derived from one species, preferably a rodent antibody or a chicken antibody, more preferably a murine antibody, while the constant domains of the antibody molecule are derived from those of a human antibody.

As used herein, the term “treatment” or “treating” of a disease is an approach for obtaining beneficial or desired results including clinical results. For purposes of the present invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods provided herein contemplate any one or more of these aspects of treatment.

As used herein, the term “administer” or “administration” refers to the act of injecting or otherwise physically delivering a substance as it exists outside the body (e.g., a formulation of the invention) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, is being prevented, administration of the substance typically occurs before the onset of the disease or symptoms thereof.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “therapeutically effective amount” or “efficacious amount” refers to the amount of a subject anti-EphA2 antibody that, when administered to a mammal or other subject for treating a disease, is sufficient to effect such treatment for the disease.

The development of next-generation antibody drugs is currently the appealing trend in cancer treatment. The present disclosure aims to develop anti-EphA2 antibody and construct antibody-drug conjugate (ADC) against a EphA2 associated cancer; particularly a pancreatic cancer. Targeting tumor-specific antigen (EphA2) with the antibody to inhibit cancer cell growth and induce cellular endocytosis, the antibody conjugated an anti-tumor compound (such as small molecule Monomethyl auristatin E (MMAE)) will result in more efficient tumor cytotoxicity.

The present invention creates anti-EphA2 antibodies, particularly, a single-chain antibody fragments (scFv) and humanized antibody, which have ability in binding to EphA2 and in inhibiting angiogenesis and cancer cell growth.

In another aspect, the present invention provides an isolated anti-EphA2 antibody or an antigen-binding portion thereof, comprising at least one of a light chain CDR1 (L-CDR1) comprising an amino acid residue of SEQ ID NO: 1, or a variant having amino acid sequence with at least 80% identity to any of SEQ ID NO: 1; a light chain CDR2 (L-CDR2) comprising an amino acid residue of DND, or a variant having amino acid sequence with at least 80% identity to any of DND; and a light chain CDR3 (L-CDR3) comprising an amino acid residue SEQ ID NO: 3, or a variant having amino acid sequence with at least 80% identity to any of SEQ ID NO: 3; and
at least one of a heavy chain complementarity determining region 1 (H-CDR1) comprising an amino acid residue of SEQ ID NO: 4, or a variant having amino acid sequence with at least 80% identity to any of SEQ ID NO: 4; a heavy chain CDR2 (H-CDR2) comprising an amino acid residue of SEQ ID NO: 5, or a variant having amino acid sequence with at least 80% identity to any of SEQ ID NO: 5; and a heavy chain CDR3 (H-CDR3) comprising an amino acid residue of SEQ ID NO: 6, or a variant having amino acid sequence with at least 80% identity to any of SEQ ID NO: 6; such that said isolated antibody or antigen-binding portion thereof binds to EphA2. Preferably, the sequence identity as mentioned above is at least 81%, 82%, 83%, 84%, 85%, 86%, 87%, 88%, 89%, 90%, 91%, 92%, 93%, 94%, 95%, 96%, 97%, 98% or 99%.

The amino acid sequences of the complementarity determining regions in light chains and heavy chains and are listed below.

CDRs of Light Chain

| L-CDR1 | L-CDR2 | L-CDR3 |
|---|---|---|
| SGSYG | DND | GSADSSTYVGM |
| (SEQ ID NO: 1) | | (SEQ ID NO: 3) |

CDRs of Heavy Chain

| H-CDR1 | H-CDR2 | H-CDR3 |
|---|---|---|
| GFTFSDYG | ISAAGSFT | ARIPSGYRAGEIYT |
| (SEQ ID NO: 4) | (SEQ ID NO: 5) | (SEQ ID NO: 6) |

In some embodiments, the isolated anti-EphA2 antibody is a monoclonal antibody, chimeric antibody, humanized antibody or human antibody. In some embodiments, the isolated anti-EphA2 antibody is a single chain antibody (such as Fv (scFv), IgG, Fab, (Fab)₂, or (scFv′)₂).

According to the invention, the embodiments of the amino acids of the light chains and the heavy chains and of the antibodies of the invention are listed below.

Embodiments of Amino Acid Sequences
of Light chains
LTQPSSVSANLGETVKIPCSGSSGSYGWYQQKSPGSAPVTLIYDNDKRPS
DIPSRFFGSTSGSTSTLTITGVQAEDEAVYFCGSADSSTYVGMFGAGTTL
TVL (SEQ ID NO: 7) (scFv SD5)
LTQPPSVSVSPGQTVRITCSGSSGSYGWYQQKSPGQAPVTLIYDNDKRPS
GIPERFSGSSSGSTSTLTISGVQAEDEADYYCGSADSSTYVGMFGGGTKL
TVL (SEQ ID NO: 8) (Humanized scFv hSD5)
Embodiments of Amino Acid Sequences
of Heavy Chains
TVTLDESGGGLQTPGGGLSLVCKASGFTFSDYGMGWVRQAPGKGLEFVAG
ISAAGSFTPYGAAVKGRATISRDNGQSTVRLQLNNLRSEDTGIYYCARIP
SGYRAGEIYTWGQGTEVIVSS (SEQ ID NO: 9) (scFv SD5)
QVQLVESGGGLVQPGGSLRLSCAASGFTFSDYGMGWVRQAPGKGLEWVAG
ISAAGSFTPYGAAVKGRFTISRDNSKNTVYLQMNSLRAEDTAVYYCARIP
SGYRAGEIYTWGQGTLVTVSS (SEQ ID NO: 10) (Humanized
scFv hSD5)

In some embodiments, the present disclosure provides a light chain comprising an amino acid sequence comprising SEQ ID NO: 7 or 8.

In some embodiments, the present disclosure provides a heavy chain comprising an amino acid sequence comprising SEQ ID NO: 9 or 10.

In further embodiments, the present disclosure provides an isolated antibody (scFv SD5), comprising a light chain having an amino acid sequence as set forth in the sequence comprising SEQ ID NO: 7 or a variant having at least 80% identity to SEQ ID NO: 7, and a heavy chain having an amino acid sequence as set forth in the sequence comprising SEQ ID NO: 9 or a variant having at least 80% identity to SEQ ID NO: 9. Preferably, the sequence identity as mentioned above is at least 90%, 91%, 92%, 93%, 94%, 95%, 96%, 97%, 98% or 99%.

In a further embodiment, the present disclosure provides a humanized antibody (Humanized scFv hSD5), comprising a light chain having an amino acid sequence as set forth in SEQ ID NO: 8 or a variant having at least 80% identity to SEQ ID NO: 8 and a heavy chain having an amino acid sequence as set forth in SEQ ID NO: 10 or a variant having at least 80% identity to SEQ ID NO: 10.

In a further embodiment, the present disclosure provides an isolated antibody (scFv SD5), comprising the following sequence:

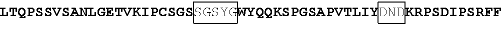

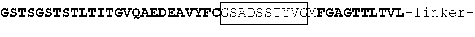

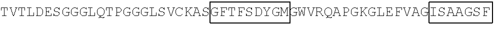

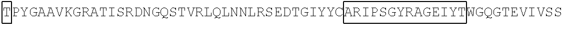

EVIVSS

The linker in the scFv can be any linker known in the art. In some embodiments, the linker has a sequence less than 10, 11, 12, 13, 14, 15, 16, 17, 18, 19 or 20 amino acids. In some embodiemnts, the linker comprises an amino acid of glycine-serine (GS) linkers, RGRGRGRGRSRGGGS or GQSSRSS.

In a further embodiment, the present disclosure provides an isolated antibody (scFv SD5), comprising an amino acid sequence as set forth in SEQ ID NO: 11 or a variant having at least 80% identity to SEQ ID NO: 11. In a further embodiment, the invention comprises a humanized antibody, comprising an amino acid sequence as set forth in SEQ ID NO: 12 or a variant having at least 80% identity to SEQ ID NO: 12.

scFv SD5
(SEQ ID NO: 11)

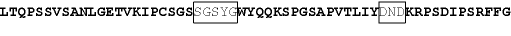

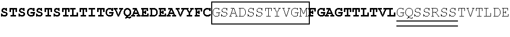

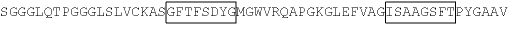

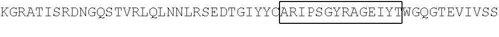

(GQSSRSS is a linker sequence)
Humanized scFv hSDS
(SEQ ID NO: 12)

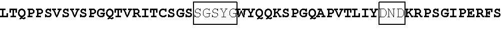

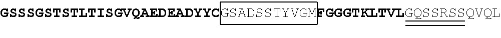

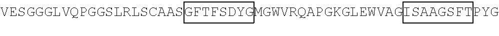

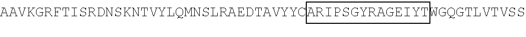

SS

Techniques for preparing monoclonal antibodies against virtually any target antigen are well known in the art. See, for example, Kohler and Milstein, Nature 256:495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice or chicken with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Various techniques, such as production of chimeric or humanized antibodies, may involve procedures of antibody cloning and construction. The antigen-binding variable light chain and variable heavy chain sequences for an antibody of interest may be obtained by a variety of molecular cloning procedures, such as RT-PCR, 5′-RACE, and cDNA library screening. The variable heavy or light chain sequence genes of an antibody from a cell that expresses a murine antibody can be cloned by PCR amplification and sequenced. To confirm their authenticity, the cloned V_{L} and V_{H} genes can be expressed in cell culture as a chimeric antibody as described by Orlandi et al., (Proc. Natl. Acad. Sci., USA, 86:3833 (1989)). Based on the variable heavy or light chain gene sequences, a humanized antibody can then be designed and constructed as described by Leung et al. (Mol. Immunol., 32:1413 (1995)).

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. Methods for constructing chimeric antibodies are well known in the art (e.g., Leung et al., 1994, Hybridoma 13:469).

A chimeric monoclonal antibody may be humanized by transferring the chicken CDRs from the heavy and light variable chains of the chicken immunoglobulin into the corresponding variable domains of a human antibody. The chicken framework regions (FR) in the chimeric monoclonal antibody are also replaced with human FR sequences. To preserve the stability and antigen specificity of the humanized monoclonal, one or more human FR residues may be replaced by the mouse counterpart residues. Humanized monoclonal antibodies may be used for therapeutic treatment of subjects. Techniques for production of humanized monoclonal antibodies are well known in the art (see, e.g., Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285; Sandhu, Crit. Rev. Biotech., 1992, 12:437; Tempest et al., 1991, Biotechnology 9:266; Singer et al., J. Immun., 1993, 150:2844).

Phage display technology can be used to produce the anti-EphA2 antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. In other embodiments, ribosome display technology can be used to produce the anti-EphA2 antibodies and antibody fragments in vitro. Various techniques have been developed for the production of antibody fragments. Traditionally, these fragments were derived via proteolytic digestion of intact antibodies.

However, these fragments can now be produced directly by recombinant host-cells, for example, using nucleic acids encoding anti-EphA2 antibodies of the present disclosure. Fab, Fv and scFv antibody fragments can all be expressed in and secreted from E. coli, thus allowing the straightforward production of large amounts of these fragments. Anti-EphA2 antibody fragments can also be isolated from the antibody phage libraries as discussed above. Alternatively, Fab′-SH fragments can be directly recovered from E. coli and chemically coupled to form F(ab′)₂ fragments. According to another approach, F(ab′)₂ fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv).

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the antibodies of the present disclosure can also be constructed in whole or in part using standard peptide synthesis well known in the art.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. Excess oxidized glutathione or other oxidizing low molecular weight compounds can be added to the refolding solution after the redox-shuffling is completed.

In addition to recombinant methods, the antibodies and variants thereof that are disclosed herein can also be constructed in whole or in part using standard peptide synthesis. Solid phase synthesis of the polypeptides can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments.

In the preliminary data, the present disclosure has isolated a specific antibody SD5 targeting the structural active site of EphA2 by phage display technology. The results have confirmed that the antibody SD5 can significantly inhibit the growth and migration of cancer cells, resulting in the molecule degradation of EphA2 and inducing the endocytosis of the targeting cell. Therefore, the antibody has the potential and value to develop into an ADC. In the preliminary experiment, the humanized antibody incorporated with MMAE named hSD5-ADC showed an excellent tumor-killing effect in vitro and in vivo, inducing the apoptosis of cancer cells. These experimental results show and support the value of continuing to develop hSD5-ADC. Moreover, the present disclosure has verified the therapeutic effect of the ADC through the complete cell and animal experiments. It is believed that this ADC drug can provide more effective and comprehensive treatment effects on an EphA2 associated cancer (particularly a pancreatic cancer and even other EphA2-associated cancers).

The present disclosure also provides an ADC, comprising the anti-EphA2 antibody of the present disclosure or an antigen-binding portion thereof and a drug-linker structure comprising an antitumor compound connected to the antibody by a linker.

The anti-EphA2 antibody of the present disclosure or an antigen-binding portion thereof can be conjugated to an antitumor compound via a linker structure moiety to prepare an anti-EphA2 antibody-drug conjugate. The antitumor compound is not particularly limited as long as it has a substituent or a partial structure that can be connected to a linker structure.

Examples of the antitumor compound include, but are not limited to, auristatins such as monomethyl auristatin E (MMAE) and monomethyl auristatin F (MMAF), vincristine, vinblastine, methotrexate, platinum-based antitumor agents (cisplatin and derivatives thereof), doxorubicin, calicheamicin, dolastatin 10, maytansinoids, a pyrrolobenzodiazepine dimer, a camptothecin derivative, duocarmycins, amanitin, daunorubicin, mitomycin C, bleomycin, cyclocytidine, and Taxol and derivatives thereof.

In the ADC of the present application, the linker structure which conjugates the anti-EphA2 antibody to the drug is not particularly limited as long as the ADC can be used. The linker structure can be appropriately selected and used according to the purpose of use.

Pharmaceutical compositions comprising an anti-EphA2 antibody or an ADC of the present disclosure are also provided.

Certain embodiments relate to a pharmaceutical composition comprising an anti-EphA2 antibody or an ADC of the present disclosure and a pharmaceutically acceptable carrier or excipient. The term “pharmaceutically acceptable carrier” is intended to include, but not limited to, a non-toxic solid, semisolid or liquid filler, diluent, encapsulating material or formulation auxiliary of any type known to persons skilled in the art. Diluents, such as polyols, polyethylene glycol and dextrans, may be used to increase the biological half-life of the conjugate.

The pharmaceutical compositions of the present invention can be formulated according to conventional methods (for example, Remington's Pharmaceutical Science, latest edition, Mark Publishing Company, Easton, U.S.A.), and may also contain pharmaceutically acceptable carriers and additives. Examples include, but are not limited to, surfactants, excipients, coloring agents, flavoring agents, preservatives, stabilizers, buffers, suspension agents, isotonic agents, binders, disintegrants, lubricants, fluidity promoting agents, and corrigents, and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such.

An embodiment is directed to a method for treating or preventing an EphA2 associated cancer in a subject, comprising administering an anti-EphA2 antibody or an ADC of the present disclosure to the subject. Alternatively, an embodiment is directed to a use of an anti-EphA2 antibody or an ADC of the present disclosure in the manufacture of a medicament for treating or preventing angiogenesis disorder in a subject.

A further embodiment is directed to a method for inhibiting an EphA2 associated cancer cell growth or EphA2 associated cancer metastasis in a subject comprising administering an anti-EphA2 antibody or an ADC of the present disclosure to the subject. Alternatively, a further embodiment is directed to a use of an anti-EphA2 antibody or an ADC of the present disclosure in the manufacture of a medicament for inhibiting EphA2 associated cancer cell growth or EphA2 associated cancer metastasis in a subject.

In one embodiment, the EphA2 associated cancer is selected from bile duct cancer, bladder cancer, brain cancer, breast cancer, cervical cancer, colon cancer, esophageal cancer, gastric cancer, gliomas, liver cancer, lung cancer, melanoma, ovarian cancer, pancreatic cancer, prostate cancer, rectal cancer, renal cancer, stomach cancer, thymus cancer, and vulvar cancer.

The present method also comprises administering an anti-EphA2 antibody or an ADC of the present disclosure concomitantly with, or subsequent to other standard therapies, wherein said standard therapy is selected from the group consisting of radiotherapy, surgery and chemotherapy.

The anti-EphA2 antibody, ADC or the pharmaceutical composition thereof may be administered intravenously, intra-peritoneally, intra-arterially, intra-thecally, intra-vesically, or intratumorally. One of ordinary skill will appreciate that effective amounts of the anti-EphA2 antibody can be determined empirically. It will be understood that, when administered to a human patient, the total daily usage of the anti-EphA2 antibody or composition will be decided by the attending physician within the scope of sound medical judgment. The specific therapeutically effective dose level for any particular patient will depend upon a variety of factors: the type and degree of the cellular response to be achieved; activity of the specific anti-EphA2 antibody, ADC or composition employed; the age, body weight, general health, sex and diet of the patient; the time of administration, route of administration, and rate of excretion of the anti-EphA2 antibody; the duration of the treatment; drugs used in combination or coincidental with the anti-EphA2 antibody, ADC or composition; and like factors well known in the medical arts.

Each of the above identified compositions and methods of treatment may additionally include an additional anti-tumor drug and the administration of an additional one or more anti-tumor drug. Anti-tumor drugs suitable for use with the present disclosure include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like. Examples of the additional anti-tumor drug include but are not limited to 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., temozolomide, dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-. alpha., etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); and 22) modulators of p53 protein function.

The present disclosure also shows that there is a connection between EphA2 levels and cancer severity; accordingly, the expression of EphA2 or a fragment thereof in high level in the biological sample as compared to a reference level of the expression of EphA2 or a fragment thereof in the control sample is indicative of prediction of a metastasis or poor prognosis. The invention unexpectedly found that the anti-EphA2 antibody of the present disclosure can be used as an indicator of a diagnosis or predication of a prognosis or an elevated risk of metastasis or future occurrence of a cancer in a subject. Accordingly, the present disclosure provides a method for detecting or diagnosing a cancer or an elevated risk of future occurrence of a cancer, or predicting a metastasis or prognosis of a cancer in a subject, comprising contacting a biological sample from a subject with an anti-EphA2 antibody of the present disclosure, quantifying the binding of EphA2 antigen in the sample to the antibody, and comparing said binding to a reference value representing binding between the anti-EphA2 antibody and the EphA2 antigen determined in samples from control subjects not afflicted with a cancer.

In one embodiment, the biological sample may be a cell, tissue, organ, organ sample, tissue biopsy, blood, plasma, serum, ascetic fluid, lymphocytes, urine, bone marrow fluid, lymphatic fluid, saliva, lachrymal fluid, mucosal fluid, amniotic fluid, or a combination thereof.

Detectable labels suitable for conjugation to antibodies and other binding reagents include radioisotopes, fluorescent labels, enzyme-substrate labels, chromogenic labels, chemiluminescent labels and colloidal gold particles.

Examples of the measurement method include, but are not limited to, a fluorescence immunoassay (FIA) method, an enzyme immunoassay (EIA) method, a radioimmunoassay (RIA) method, a Western blotting method, dot blot, an immunohistochemical assay, Fluorescence Activated Cell Sorter (FACS), in vivo imaging and a radio-imaging assay.

The present disclosure further provides a method for monitoring the progression of a cancer in a subject already diagnosed with a cancer. In some embodiments, monitoring can be used to evaluate whether a particular treatment is successful.

In some embodiments, monitoring cancer progression comprises determining a first level of EphA2 or a fragment thereof in a first biological sample obtained from a subject diagnosed with a cancer by the anti-EphA2 antibody of the present disclosure; and determining a second level of EphA2 or the fragment thereof in a second biological sample obtained from the subject by the anti-EphA2 antibody of the present disclosure after a predetermined period of time; comparing the first and second levels of EphA2 or the fragment thereof; wherein a higher level of EphA2 or the fragment thereof in the second sample compared to the first sample is indicative of disease progression and worsening. Similarly, a lower level of EphA2 or the fragment thereof in the second sample compared to the first sample is indicative of improvement.

The diagnostic methods of the present disclosure may be combined with the known diagnostic methods for a cancer.

Another aspect of the present disclosure encompasses a kit for detecting or diagnosing a cancer or an elevated risk of future occurrence of a cancer, or predicting a metastasis or prognosis of a cancer, or monitoring cancer progression in a subject. It is typically in a package which contains all elements, optionally including instructions. The package may be divided so that components are not mixed until desired. Individual components may be separately packaged within the kit. The kit may contain reagents necessary to detect the expression level of a marker gene. A kit is any manufacture (e.g., a package or container) comprising at least one reagent, e.g., an antibody reagent(s), for specifically detecting and/or measuring the expression level of a marker gene in a sample.

A variety of kits having different components are contemplated by the current disclosure. Generally speaking, the kit will include the means for quantifying EphA2 or more biomarkers in a subject. In another embodiment, the kit will include means for collecting a biological sample, means for quantifying EphA2 or more biomarkers in the biological sample, and instructions for use of the kit contents. In certain aspects, the kit comprises a means for quantifying the amount of a biomarker. In further aspects, the means for quantifying the amount of a biomarker comprises reagents necessary to detect the amount of a biomarker.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLE

### Materials and Methods

### Cell Culture and Animal Immunization

The human PAAD cell lines AsPc-1, BxPc-3, Panc-1, and Mia PaCa-2 were purchased from the American Type Culture Collection (ATCC) (Manassas, VA, USA). All cell lines were cultured according to ATCC standard protocols and incubated at 37° C. in a humidified atmosphere of 5% CO2. Female white leghorn (Gallus domesticus) chickens and nonobese diabetic mice with severe combined immunodeficiency (NOD/SCID) were purchased from the National Laboratory Animal Center, Taiwan, and were maintained in the animal facility of Taipei Medical University.

### Bioinformatics Analysis

To investigate the association between EphA2 gene expression and PAAD occurrence, we used the Gene Expression Profiling Interactive Analysis (GEPIA; http://gepia.cancer-pku.cn) database to analyze the difference in EphA2 expression in clinical cancer samples and normal samples and to analyze the correlation between EphA2 expression and the PAAD survival rate (Nucleic Acids, Res 45 (W1): W98-W102 (2017)).

### Antibody Library Construction and Biopanning

On the basis of the complex protein structure of ephrinA1-EphA2 (Protein Data Bank [PDB] ID: 3CZU), we designed a peptide immunogen for the EphA2 target molecule; the peptide immunogen (EphA2pep) contains Epitope 1, GWDLMQNIMNDMPIYMYSV, and Epitope 2, VSSDFEARHV, which were linked together by using a linker GGGGGGS. EphA2pep contains six consecutive repetitive sequences. After gene synthesis (GENEWIZ), it was constructed on a pET21a vector (Novagen) and transformed to the Escherichia coli BL-21 (DE3) strain for its expression as a recombinant protein. After purification by using Ni²⁺-charged sepharose (GE Healthcare Life Sciences), the recombinant EphA2pep protein was used for animal immunization. Female white leghorn chickens were immunized through intramuscular injection of 50 μg of recombinant EphA2pep protein mixed with an adjuvant each time. During the immunization, we used Freund's complete adjuvant (Sigma-Aldrich) the first time and Freund's incomplete adjuvant (SigmaAldrich) all other times. The immunization schedule comprised four immunizations performed at intervals of 7 days. The spleens of the chickens were harvested 7 days after the final immunization to construct an scFv antibody library. The library was constructed in accordance with published protocols, with minor modifications (J Immunol Methods., 242 (1-2): 159-181 (2000)).

For panning, the recombinant EphA2 protein was precoated onto the well of a microtiter plate at 4° C. overnight. On the next day, the EphA2 protein was removed, and the well was blocked with 3% BSA at room temperature for 1 h. Then, the recombinant library phage solution (10¹¹ phage particles) was added to the well and incubated at room temperature for 2 h. Unbound phages in the supernatants were removed, and the well was washed through pipetting with phosphate-buffered saline with 0.05% Tween 20 (PBST) 10 times. Subsequently, bound phages were eluted with 0.1 M HCl-glycine (pH 2.2)/0.1% BSA elution buffer and neutralized with 2 M Tris base buffer. The eluted phages were immediately used to infect the E. coli ER2738 strain for recombinant phage amplification. Amplified phages were precipitated and recovered through a previously method (Proc Natl Acad Sci., 88 (18): 7978-7982 (1991)) and were used in the next round of panning. The panning procedure was repeated four times to efficiently enrich anti-EphA2 binding phages. After panning, the total library DNA was purified and transformed into the E. coli strain TOP 10F′ (Invitrogen, a nonsuppressor strain) for scFv expression. The expressed scFv was further purified with Ni²⁺-charged sepharose in accordance with the manufacturer's instructions (GE Healthcare Life Science).

### Sequence Analysis

To sequence the scFv clones of interest, we used the ompseq primer (5′-AAGACAGCTATCGCGATTGCAGTG-3′) complementary to the outer membrane protein A (ompA) signal sequence upstream of the light chain variable region. Next, the website International ImMunoGeneTics information system/V-QUEry and Standardization (http://imgt.org) were used to compile and analyze the sequence data on the basis of the germline genes.

### Enzyme-Linked Immunosorbent Assay

The recombinant EphA2 protein (0.5 ug/well) was coated onto the wells of the microtiter plate at 4° C. overnight. The wells were blocked with 5% skim milk, and scFv or phage was then added to the wells at room temperature for 1 h. After the wells were washed with PBST, the bound scFv or phage was then detected and developed using horseradish peroxidase (HRP)-conjugated goat anti-chicken light chain antibodies (Bethyl Laboratories) or HRP-conjugated anti-M13 antibodies (GE Healthcare Life Science). Finally, the substrate 3,5,5-tetramethubezidine dihydrochloride (TMB) was added for signal development. The reaction was stopped by adding 1 N HCl, and absorbance was measured by determining optical density (OD) at 450 nm.

### Western Blotting and Immunoprecipitation Assay

The recombinant EphA2 protein was transferred onto nitrocellulose membranes (GE Healthcare Life Sciences) after electrophoresis using sodium dodecyl sulfate-polyacrylamide gel (SDS-PAGE) or nativepolyacrylamide gel (Native-PAGE), and the membranes were incubated with using purified scFv antibodies to determine binding reactivity. The membranes were blocked with 5% skim milk and then incubated with scFv at room temperature for 1 h. After washing with PBST, the membranes were detected and developed using HRP-conjugated goat anti-chicken light chain antibodies. Finally, the 3,3′-diaminobenzidine substrate was added for color development until the desired color intensity was reached.

For the immunoprecipitation assay, 300 μg of each PAAD cell lysate was incubated with 50 μg of antiEphA2 scFv (scFv was fused with a His tag) at 4° C. overnight. On the next day, 30 μL of Ni²⁺-charged sepharose was added to the mixture and incubated for 1 h at 4° C. After three rounds of washing with NiNTA wash buffer (50 mM NaH₂PO₄, 300 mM NaCl, and 10 mM imidazole, pH 8.0), the scFv-bound sepharose beads were resuspended in 50 μL of PBS buffer. Subsequently, the sepharose solution was denatured at 95° C. for 10 min and analyzed using SDS-PAGE. After the proteins were transferred to a polyvinylidene fluoride membrane, the membrane was detected using the anti-EphA2 antibody (R&D Systems) and anti-His (Proteintech Group) antibody at 4° C. overnight. On the next day, after washing with PBST, the membrane was incubated with HRP-conjugated secondary antibody (Jackson ImmunoResearch Laboratories). Finally, the chemiluminescence substrate was added for luminal signal detection.

### Flow Cytometry Analysis

Four PAAD cells AsPc-1, BxPc-3, Panc-1, and Mia PaCa-2 with endogenous EphA2 molecule expression, were analyzed through flow cytometry to determine the binding reactivity of indicated scFvs. Freshly prepared cancer cells were harvested and washed twice with PBS. Then, individual scFv was added and incubated at room temperature for 1 h. Bound scFv was visualized using goat anti-chicken light chain antibodies and donkey anti-goat antibodies conjugated with fluorescein isothiocyanate (FITC; Jackson ImmunoResearch Laboratories). An irrelevant scFv was used as the negative control, and commercially available goat anti-EphA2 antibodies were used as the positive control (R&D Systems) in the assay. The results were analyzed using a FACS can flow cytometer (BD Biosciences, Systems and Reagents).

To detect the binding specificity of scFv, 293T cells were transformed with EphA1-A8 plasmids individually for overexpression of different EphA molecules. The cell line was freshly prepared and washed with FACS buffer (2% FBS in PBS). The cells were seeded at 1×10⁵ cells/well into a 96-well Ubottom plate and incubated with scFv for 1 h at 4° C. After the plate was washed with FACS buffer, the anti-hemagglutinin (HA) antibody was added for scFv binding detection (scFv was fused with a HA tag) and subsequently developed using an FITC-conjugated secondary antibody. Finally, the cell binding signal was analyzed using a FACS can flow cytometer.

### Cell Proliferation Assay

PAAD cell proliferation was measured using a 3-(4,5-dimethylthiazol-2-yl)-5-(3-carboxymethoxyphenyl)-2-(4-sulfophenyl)-2H-tetrazolium (MTS) Cell Proliferation Assay Kit (Promega). The cells were seeded in a 96-well culture plate at a density of 5000 cells/well for attachment. Then, scFv at various concentrations was added to the cell culture and incubated for 5 days. Finally, MTS and phenazine methosulfate solutions were added and incubated for 90 min for development. After the SDS reagent was added to stop the reaction, the absorbance of each well was measured by determining the OD at 490 nm.

### Cell Migration and Scratch Wound Healing Assay

PAAD cells were seeded at 5×10⁴ cells/well in a 24-well Transwell cell migration plate (Corning) and incubated with scFv for 2 days. In the assay, recombinant ephrin-A1 (Sino Biological) was added as a positive control. After scFv treatment, the cells were fixed with frozen 100% methanol for 10 min and stained with 0.01% crystal violet for 1 h at room temperature. After the pate was washed with ddH₂O, the upper-layer cells were removed using cotton swabs. Cell staining was imaged using a microscope and analyzed using ImageJ. For the scratch wound healing assay, PAAD cells were seeded in a 6-well culture plate. The seeded cells in the well were scratched with a pipette tip to simulate a wound. After treatment with scFv and incubation for 36 or 72 h, the cells were imaged using a microscope. The wound areas were analyzed using ImageJ.

For the scratch wound healing assay, PAAD cells were seeded in a 6-well culture plate. The seeded cells in the well were scratched with a pipette tip to simulate a wound. After treatment with scFv and incubation for 36 or 72 h, the cells were imaged using a microscope. The wound areas were analyzed using ImageJ.

### Molecule Signaling

To determine molecule signaling in BxPc-3 and Mia PaCa-2 cells, the cells were cultured in a 6-well culture plate and treated with scFv antibodies at the indicated concentrations for 24 h. The cells were collected and lysed using lysis buffer [50 mM Tris-HCl (pH 7.5), 50 mM NaCl, 5 mM ethylenediaminetetraacetic acid, and 1% Triton X-100], and a mixture of proteinase inhibitors (Roche Applied Science) was added. The protein concentration of the cell lysate was measured through the Coomassie Plus (Bradford) Protein Assay (Thermo Fisher Scientific). Samples were run on reducing SDS-PAGE for Western blotting analysis and were detected using antibodies against p-EphA2, EphA2, p-AKT, AKT, p-ERK, ERK, p-FAK, FAK, pSTAT3, STAT3 (Cell Signaling Technology), and β-actin (GeneTex).

### Antibody Internalization Assay

BxPc-3 cells were seeded on cover glasses placed in the wells of a 6-well culture plate. The BxPc-3 seeded glass slides were incubated with ephrin A1-Fc (Sino Biological) or IgG hSD5 for 1 h at 37° C. or 4° C. After washing with PBS, the cells were fixed with 100% ice methanol for 10 min. Next, the cells were stained with an FITC-conjugated anti-human Fc antibody and were subsequently mounted with ProLong Diamond Antifade Mountant comprising 4′,6-diamidino-2-phenylindole for nuclear counterstaining (Invitrogen). The cells were imaged using a confocal microscope (Leica Microsystems).

### Tumor Xenograft Model

The freshly prepared BxPc-3 and Mia PaCa-2 cancer cells were harvested during the log growth phase and resuspended in PBS for tumor implantation. Each NOD/SCID mouse was subcutaneously inoculated with cancer cells (5×10⁶ to BxPc-3 and 1×10⁷ to Mia PaCa-2) for tumor formation. The tumor size was measured twice weekly, and the volume was calculated as follows: V=0.5lw², where l=the length and w=the width. When the tumor size was approximately 100 mm3, animals were divided into groups that received (a) vehicle alone through intravenous injection (i.v.) once weekly (qwk), (b) control human IgG1 at 20 mg/kg through i.v. qwk, (c) and (d) hSD5 IgG1 at 2 or 20 mg/kg through i.v. qwk, (e) and (f) gemcitabine at 20 or 100 mg/kg through i.v. twice weekly (biw), or (g) hSD5 IgG1 at 2 mg/kg through i.v. qwk combined with gemcitabine at 20 mg/kg through i.v. biw. At the end of the experiment, the antitumor effects were quantified by dividing the tumor volumes in the treatment groups by those in the control groups and multiplying them by 100 to represent tumor growth inhibition (TGI; %). The mice were also examined frequently for overt signs of adverse drug-related side effects.

### Immunohistochemical Staining

The pancreatic tissue microarray slide (US Biomax, PA483e) was used to detect and analyze the EphA2 expression of clinical samples. In the xenograft animal model, the excised BxPc-3 and Mia PaCa-2 tumors were fixed in formalin, embedded in paraffin, and sliced for immunohistochemical staining (IHC). Commercial antibodies (Cell signaling, Dako; Agilent Technologies; and Abcam) were used for staining the EphA2 molecule, cell proliferation marker Ki-67, and apoptosis marker cleaved caspase 3. The effects of staining were observed using a Zeiss Axioskop-2 microscope (Carl Zeiss).

### Interaction Residue Definition

We used peptide enzyme-linked immunosorbent assay (ELISA) to identify the designed epitope on EphA2; the BSA-conjugated peptides EphA2pep_P1 (BSA-CGGGWDLMQNIMNDMPIYMYSV) and EphA2pep_P2 (BSA-CGGGGGGSVSSDFEARHV) each represent the two segments of the linear epitope designed on EphA2. EphA2pep_PIP2 (BSA-CGGGWDLMQNIMNDMPIYMYSVGGGGGGSVSSDFEARHV) represents the two linear epitope sequences connected by a linker. These peptides were synthesized and conjugated with BSA (Kelowna International Scientific). The BSA-conjugated peptides were individually coated on a 96-well microplate at 4° C. overnight. After the wells were blocked with 3% BSA, IgG hSD5 was added and incubated. Next, an HRP-conjugated anti-HA tag antibody (Cell Signaling Technology) was used to detect bound scFv. Finally, the TMB substrate was added for development, and the reaction was stopped by adding 1 N HCl. Absorbance was measured by determining the OD at 450 nm. A dot blot assay was used to detect the binding of IgG hSD5 to the synthesized peptides; 1 mL of individual peptides (1 mg/mL; in triplicate) were dropped on the NC membrane and maintained at RT for complete absorption. Blocking with 3% BSA was performed at RT for 1 h. Then, IgG hSD5 (10 μg/mL) was added, and the reaction was allowed to proceed for 1 h at RT. After the membrane was washed, the HRP-conjugated anti-HA tag antibody was added, and the reaction was allowed to proceed for 1 h at RT. Lastly, DAB was used to initiate the coloration reaction.

### Statistical Analysis

The data are presented as mean±standard error of the mean and were analyzed using GraphPad Prism (GraphPad Software). Statistical comparisons between groups were performed using one-way analysis of variance, which was followed by a post hoc Tukey's honest significant difference test. P values lower than 0.05 were considered significant.

### Example 1. Cancer Types with High EphA2 Gene Expression were Analyzed Using GEPIA Database. Cancer Samples are Red Dots, and Normal Samples are Green Dots

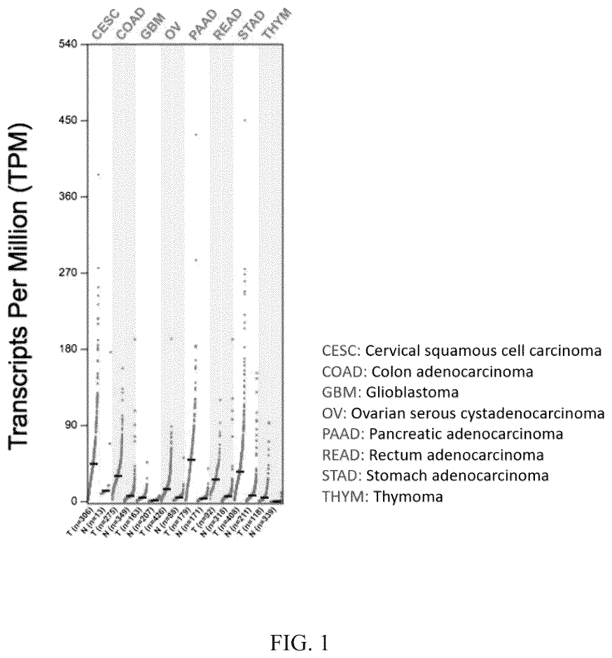

The GEPIA database was used to analyze cancer types with high expression of the EphA2 gene, including cervical squamous cell carcinoma, colon adenocarcinoma, glioblastoma, ovarian serous cystadenocarcinoma, rectum adenocarcinoma, stomach adenocarcinoma, and thymoma. The red dots represent the cancer samples, whereas the green dots represent the normal samples. (FIG. 1)

It was discovered that the cancer types with high expression of the EpHA2 gene were cervical squamous cell carcinoma, colon adenocarcinoma, glioblastoma, ovarian serous cystadenocarcinoma, rectum adenocarcinoma, stomach adenocarcinoma, and thymoma. The involved organs of the digestive tract were the stomach, pancreas, and colorectum, suggesting a correlation between the expression of EphA2 and the digestive organs. The involved organs of the digestive tract were the stomach, pancreas, and colorectum, suggesting a correlation between the expression of EphA2 and the digestive organs.

### Example 2. Analysis and Evaluation of the Correlation Between the EphA2 Expression and Pancreatic Cancer (PAAD)

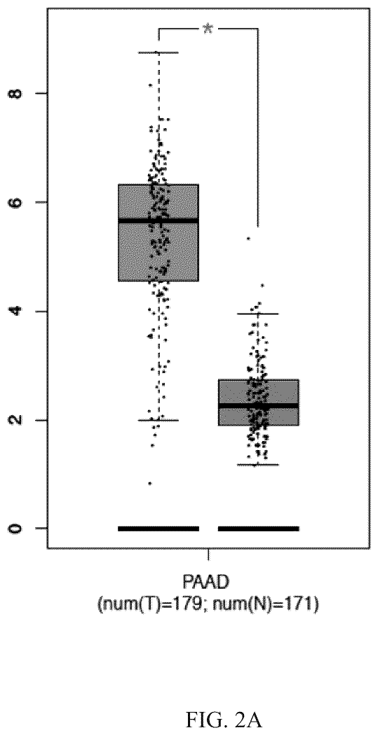

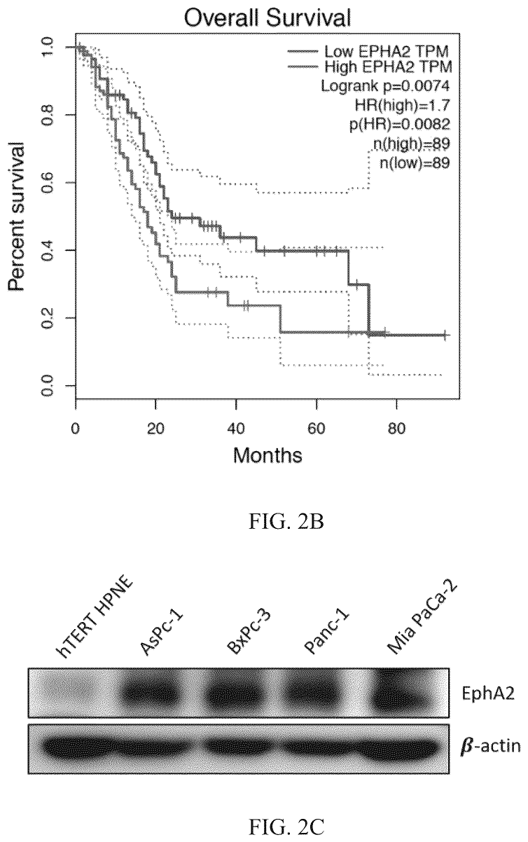

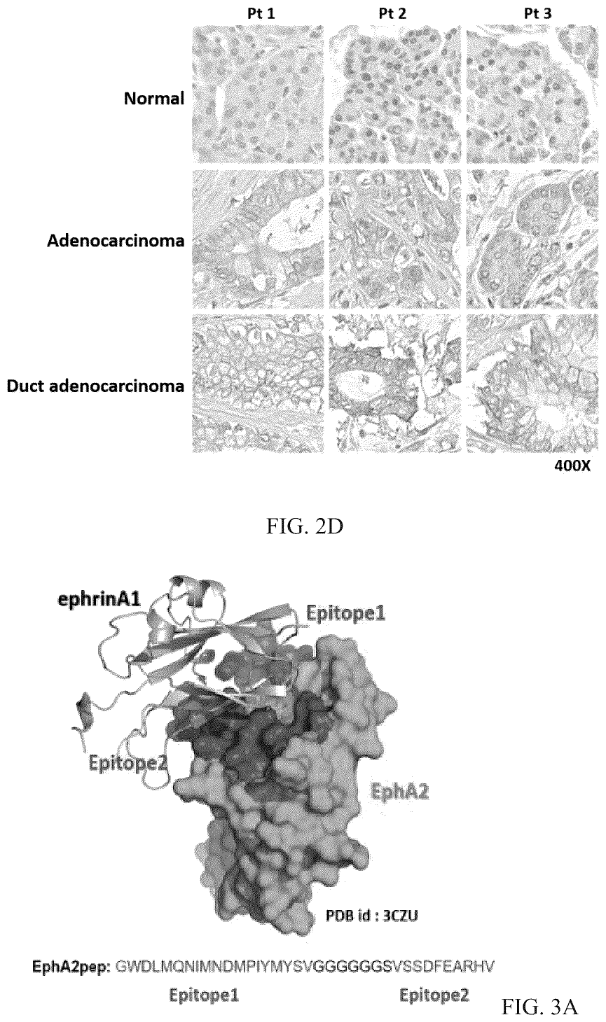

Differences in the expression of EphA2 gene in pancreatic cancer samples (red bar) and normal samples (grey bar) was analyzed using GEPIA database. (FIG. 2A). The red bar represents the cancer samples (n=179), and the gray bar represents normal samples (n=171). It was found that the genetic expression of EphA2 exhibited a meaningful increase in the PAAD samples

FIG. 2B illustrates the correlation between EphA2 gene expression and survival in pancreatic cancer patients. Through the analysis of survival correlation, it was discovered that patients with high EphA2 gene expression (red line) have lower survival rate than low EphA2 gene expression (blue line). (FIG. 2B)

To analyze the expression of EphA2 molecules, four PAAD cell lines AsPc-1, BxPc-3, Panc-1, and Mia PaCa-2 and one normal pancreatic endothelial cell line hTERT-HPNE to compare the expression of EphA2 in cancer cells were used by western blotting. (FIG. 2C) All four PAAD cell lines exhibited higher expression of the EphA2 protein than that of the control hTERT-HPNE cell line.

The IHC staining with tissue array was used to analyze the expression of EphA2 molecules in clinical pancreatic cancer tissue and normal pancreatic tissue. (FIG. 2D). It was discovered that higher levels of EphA2 in the cell membranes of the clinical PAAD tissue specimens than in the normal pancreatic tissues. In the specimen stained in FIG. 2D, a tissue microarray glass slide containing 38 pancreatic carcinomas and 8 normal pancreatic tissues was used for IHC analysis. Of the 38 cases, 37 were adenocarcinomas with high, moderate, and low levels of differentiation in terms of histopathology. Only one case was classified as squamous cell carcinoma. Careful microscopical investigation by a pathologist revealed that the EphA2 molecules in 34 of the 37 adenocarcinomas were strongly stained in a diffuse, cytoplasmic staining pattern, whereas those for squamous cell carcinoma were entirely negative; focal staining was observed in the acinus areas, but not in the ductal compartment of five of the eight normal pancreatic tissues. In two of the three adenocarcinomas with low levels of differentiation, diffuse, cytoplasmic, and focal and typical membrane staining patterns were observed, suggesting that EphA2 is a membranous biomarker often identified in cancer cells with extremely high histopathological grading.

The results indicate that the EphA2 biomarker can be cancer-specific and can be applied for IHC staining for diagnosis and prognostic purposes for adenocarcinomas of the pancreas; it presents a characteristic cytoplasmic staining pattern in most carcinoma cases. For poorly differentiated adenocarcinomas of the pancreas, EphA2 tends to translocate to the cell surface or cell membrane of high-grade cancer cells.

### Example 3. Characterization of Anti-EphA2 scFvs Isolated Using Phage Display Technology

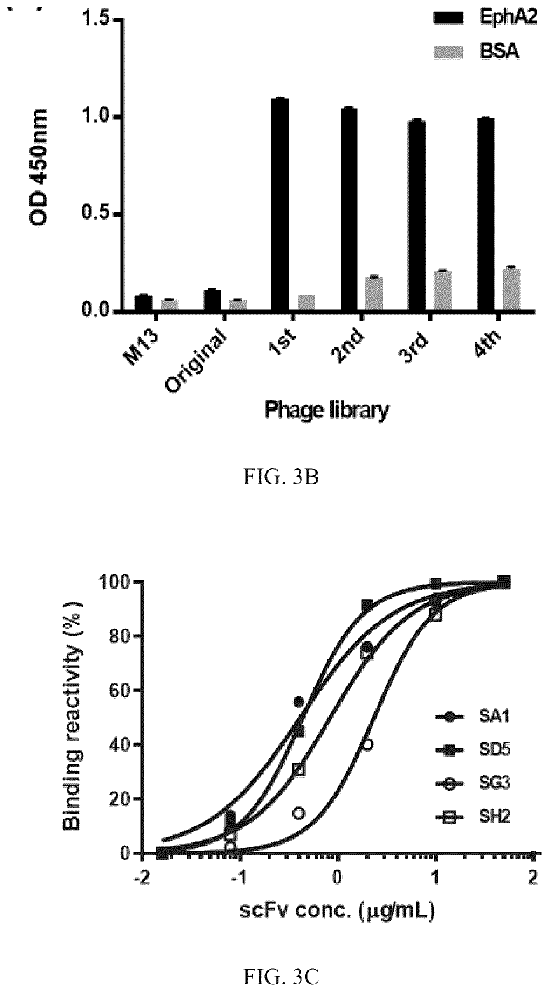

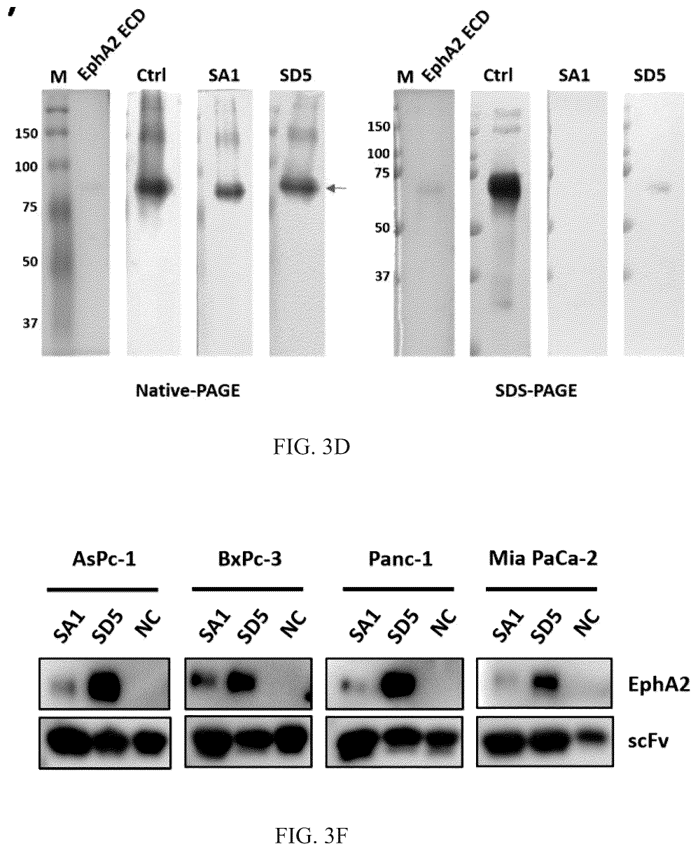

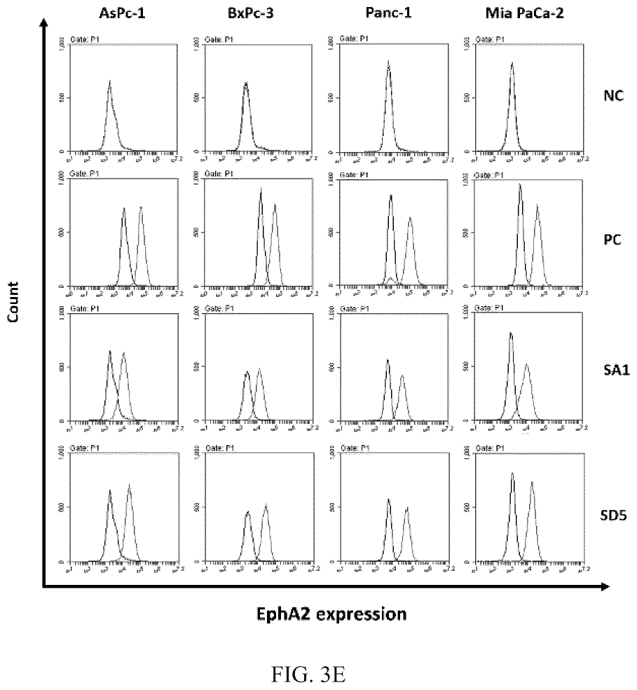

To allow the isolated antibodies to recognize the activation site on EphA2, discontinuous fragments as immunogens was designed on the basis of a published EphA2 complex structure. According to the X-ray crystal structure of PDB id: 3CZU, the design of the discontinuous antigenic peptide in the active site of EphA2 is carried out. Ephrin-A1 is gray, EphA2 is blue-green, and the red part is two antigenic peptides (Epitope1 and Epitope2) located on EphA2. EphA2pep is designed by linking two antigenic peptides. (FIG. 3A) As shown in FIG. 3A, the structure of the complex protein (PDB ID: 3CZU) formed by EphA2 (blue-green) and ephrin-A1 (gray) was analyzed. The binding site of ephrin-A1 was expanded to the range of 5 Å and determined the amino acids in the EphA2 molecule that may result in interactions. On the basis of this information, a peptide sequence with discontinuous fragments (red: Epitope 1 and Epitope 2) was created, and a linker sequence (GGGGGGS) was used to link EphA2pep: GWDLMQNMNDMPIYMYSVGGGGGGSVSSDFEARHV. After expressing the 6-repetition peptide sequences as a recombinant protein, the chickens were then immunized. Subsequently, a highly complex library of scFv antibodies and isolated specific antibodies were constructed by using phage display technology. It was found that the number of bound phages increased by approximately 60-fold between the first and last rounds (data not shown), indicating that the specific binding strains were enriched by panning.

The phage ELISA was used to test the amplified phage antibody library after each round of panning, with “original” representing the original antibody library, and “M13” representing the wild-type phage. (FIG. 3B) As shown in FIG. 3B, in phage ELISA, the amplified phage library obtained after the first round of panning already contained enriched specific strains that exhibited considerable binding reactions to EphA2 compared with the original antibody library (original) and wild-type M13 phage.

Four representative scFvs were serially diluted to test the percentage of binding to EphA2 molecules. (FIG. 3C) After gene sequencing, we serially diluted the four isolated representative scFvs to test the binding reaction to EphA2. The EC50 values of scFv SA1 and SD5 were similar at 1.7 and 1.8 nM, and the EC50 values of Page 5/25 scFv SG3 and SH2 were 33.6 and 94 nM, respectively; the difference in binding capacity resulted from the interaction of the CDR sequences of different antibodies on the EphA2 molecule.

The scFv SA1 and SD5 were used to identify EphA2 protein under reducing (SDS-PAGE) and non-reducing (Native-PAGE) conditions. Ctrl is the result of using commercial Ab for detection. (FIG. 3D) The scFv SA1 and SD5 with the best EC50 values were selected for subsequent testing. By using NativePAGE (red arrow), it was determined that scFv SA1 and SD5 can recognize EphA2 in the native form, but not in the denatured form, which indicates that the epitope recognized by scFvs is a conformational epitope.

The scFv SA1 and SD5 were used to identify endogenous EphA2 molecules on 4 pancreatic cancer cell lines by flow cytometry analysis. PC uses a commercial anti-EphA2 antibody, and NC is the control group without adding the antibody. (FIG. 3E) The results indicate that the scFvs exhibited a significant binding response to the EphA2 molecules on cancer cells.

To further explore the ability of the antibody to bind to the endogenous EphA2 molecule, the scFv SA1 and SD5 were used to pull down natural EphA2 molecule in the lysate of 4 pancreatic cancer cell lines by immunoprecipitation assay; NC was an irrelevant scFv did not respond. (FIG. 3F) Compared with the irrelevant scFv in the control group, which did not bind to EphA2, both scFv SA1 and SD5 captured free EphA2 molecules from the lysates of the four strains of PAAD cells, and the binding effect of scFv SD5 was superior to that of SA1.

### Example 4. Inhibition Effect of Isolated scFvs on the Proliferation and Migration of PAAD Cells

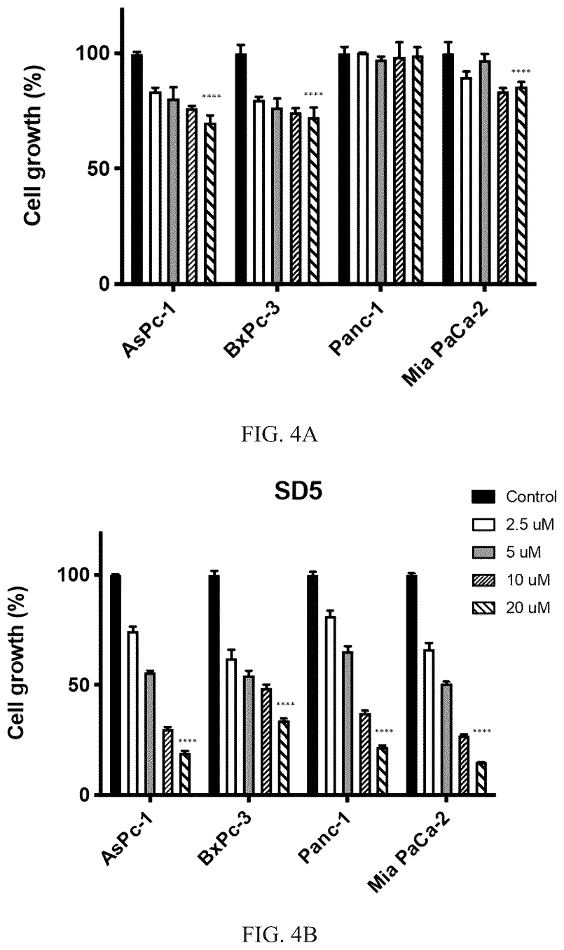

As shown in FIGS. 4A-4B, a cell survival analysis assay (MTS assay) was conducted to observe the growth inhibitory effect of scFv SA1 and SD5 on the four PAAD cell lines by adding different concentrations of antibodies to the cell culture. At indicated concentrations, scFv SA1 and SD5 interacted with the cancer cells for 5 days, and the effects on the growth of cancer cells were observed. By Day 5 of the antibody reaction, it was found that scFv SA1 exhibited an approximately 18%-24% inhibitory effect on the cancer cell lines AsPc-1 and BxPc-3. However, at a concentration of 20 mM, scFv SD5 inhibited the growth of three cell lines AsPc-1, Panc-1, and Mia PaCa-2, by approximately 80%. Additionally, it inhibited the growth of the cell line BxPc-3 by 58.5%. Dose-dependent responses were observed at different antibody concentrations; this result indicates that the binding of scFv SD5 to EphA2 on the surface of PAAD cells can inhibit the growth of cancer cells.

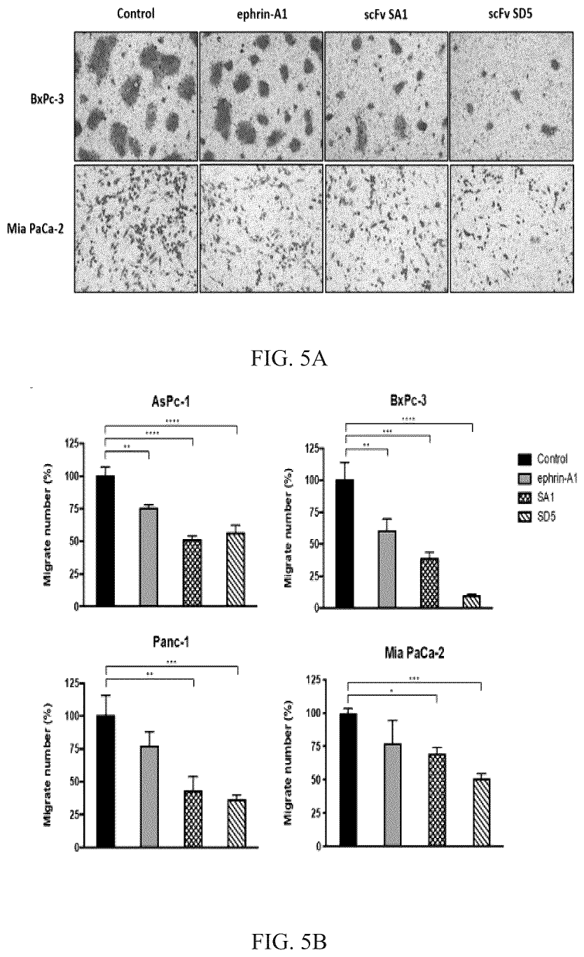

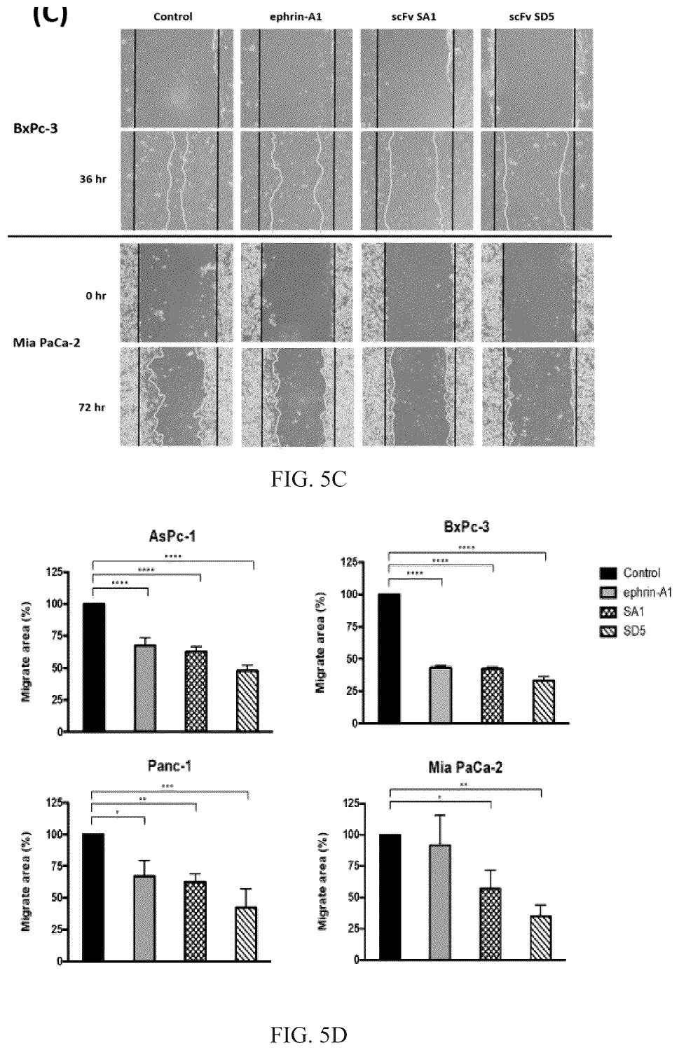

Since the molecular regulation of EphA2 has been demonstrated to promote the migration of cancer cells, both the transwell migration assay and wound healing assay were used to determine whether scFv SA1 and SD5 inhibit the migration of PAAD cells. The results of the transwell migration assay are presented in FIG. 5A. The experimental responses of the cancer cells BxPc-3 and Mia PaCa-2 revealed that after treatment with scFv SA1 and SD5 at the dose of 20 mM for 2 days, the number of migrated cancer cells were reduced effectively. The same concentration of ephrin-A1 treatment was given as a control group in the experiment. (FIG. 5A)

The quantitative percentage illustrates the migration inhibition effect of scFvs on the four pancreatic cancer cells in the transwell migration assay. The experimental results for scFv SD5 were better than those for scFv SA1. The scFv SD5 can inhibit the migration of the cancer cell lines Panc-1 and BxPc-3 by 65% and 91%, respectively. (FIG. 5B)

The results of the wound healing assay are presented in FIG. 5C. Based on the experimental reactions of the cancer cells BxPc-3 and Mia PaCa-2, the treatment of scFv SA1 and SD5 inhibited the migration of cancer cells after 36 and 72 h of treatment with scFv. The same concentration of ephrin-A1 treatment was given as a control group in the experiment. (FIG. 5C)

The quantitative percentage illustrates the migration inhibition effect of scFvs on the four pancreatic cancer cells in the wound healing assay. The experimental results for scFv SD5 are superior to those of scFv SA1; scFv SD5 exhibited higher reactivity to the cancer cell lines Mia PaCa-2 and BxPc-3, and the inhibitory effects were 65% and 67%, respectively. Therefore, we selected scFv SD5 for the subsequent experiments. (FIG. 5D)

### Example 5. Binding Specificity of Humanized Antibody Humanized Antibody hSD5 to EphA2 and Induced Tumor Suppressor Signaling

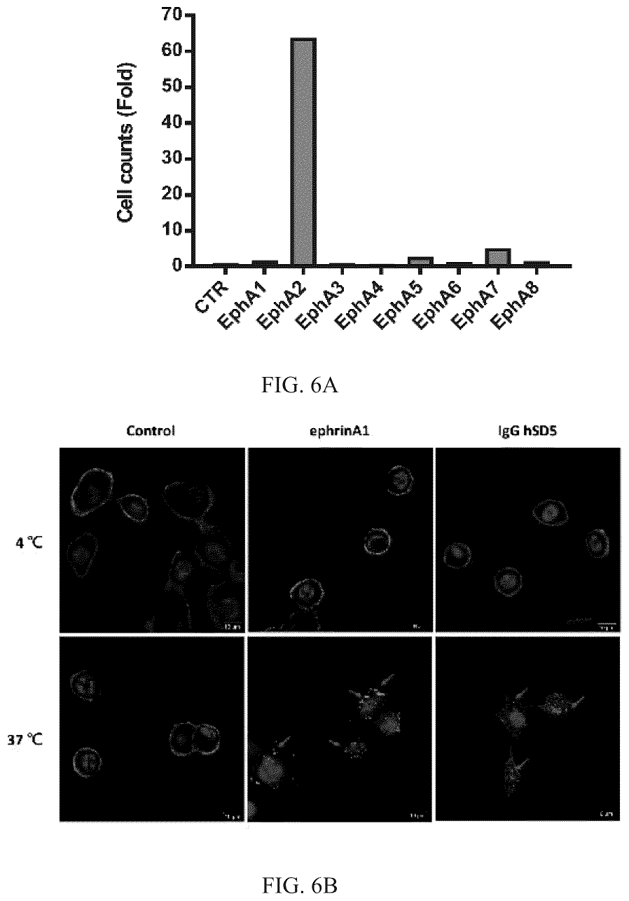

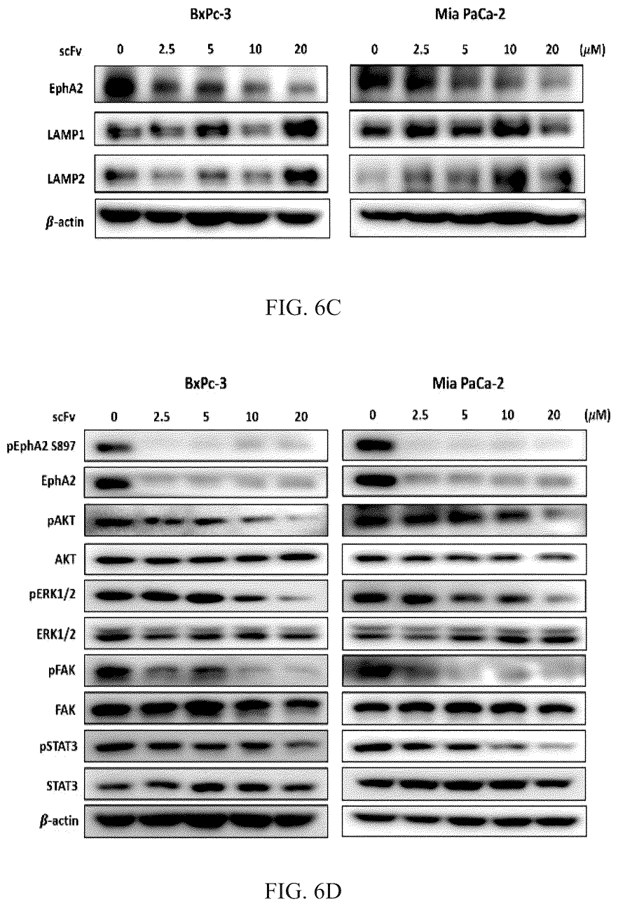

FIG. 6A demonstrates the binding reactivity of humanized scFv hSD5 to different overexpressed Eph family protein (EphA1-A8) cells. To improve the clinical applicability of the antibodies, the humanization of chicken derived scFv SD5 was performed. Since the Ephs family molecules (EphA1-A8) have numerous roles in human cell physiology, the specificity of the isolated antibodies for EphA2 molecules must be determined. By using cells with overexpression of EphA1-A8 molecules, it was demonstrated that humanized scFv hSD5 specifically binds to EphA2 molecules and does not exhibit cross-binding reactions to other family proteins. (FIG. 6A)

FIG. 6B demonstrates the endocytosis of humanized IgG hSD5 treated on pancreatic cancer cells BxPC3. The complete IgG hSD5 were expressed to conduct an experiment on endocytosis. After the PAAD cells BxPc-3 were treated with IgG hSD5, they were incubated at 4° C. and 37° C. for 1 h for observation. The ligand ephrin-A1 of EphA2 was used as a positive control for comparison. Control Ab was a commercial anti-EphA2 IgG antibody that could not induce endocytosis. The red arrows show that ephrin-A1 and IgG hSD5 are endocytosed from the cell membrane into the cytoplasm after targeting EphA2 molecule.

Incubating the cells at 4° C. would cause the cells to enter a resting state. Unlike in the control IgG group, the reactions of the antibodies for recognizing EphA2 occurred on the cell membrane at both 4° C. and 37° C. When the IgG antibody hSD5 was administered at 37° C., the antibodies entered into the cells through the cell membrane into the cytoplasm (indicated by the red arrow) through endocytosis. These experimental results were similar to the results of the experiments in which ephrinA1 (EphA2 ligand) was administered. (FIG. 6B) Using the Biolayer interferometry analysis, we analyzed the kon and koff parameters of IgG hSD5 targeting the EphA2 protein; the calculated affinity (KD) of IgG hSD5 was 2.06 nM.

As shown in FIG. 6C, Changes in molecular signaling in cancer cells after the administration of scFv hSD5 were observed. The cancer cells BxPc-3 and Mia PaCa-2 degraded EphA2 molecules 6 h after the administration of scFv and exhibited a dose-dependent response. The lysosomal-associated proteins LAMP1 and LAMP2 also exhibited upregulated levels in the two cancer cell lines, which means that scFv hSD5 may enter the cell through endocytosis after acting on EphA2 and that the lysosome is involved in the degradation of the protein. (FIG. 6C)

After the cancer cells BxPc-3 and Mia PaCa-2 were treated with different concentrations of humanized scFv hSD5 for 24 hours, the cell's molecular signaling was analyzed. As shown in FIG. 6D, EphA2 was almost completely degraded, and the amount of pEphA2 also decreased. Regarding the signals pERK and pAKT, which are associated with cancer cell proliferation and metastasis, also exhibited a similar decrease. The signals related to cancer cell survival and adhesion, a dose-dependent decrease was observed for both signals of pSTAT3 and pFAK in both cancer cell lines. Treatment with scFv hSD5 resulted in similar changes in the molecule signaling of both cancer cell lines. (FIG. 6D)

The above example revealed that the binding of the antibody hSD5 to the EphA2 molecule on the surface of cancer cells causes the degradation of the EphA2 molecule and induces endocytosis of the cell, which enables the entry of the antibody molecule into the cytoplasm (FIG. 6D); this is similar to the process of antibodies binding at the EphA2 active site and producing an ephrinA1 targeting-like response. This observation indicates that hSD5 can be developed into an antibody drug conjugate. In preliminary experiments, it was found that labeling hSD5 with the small molecule of MMAE is effective in inducing apoptosis in cancer cells.

### Example 6. In Vivo Tumor Growth Inhibitory Effect of Humanized IgG hSD5 on BxPc-3 Xenograft Mice

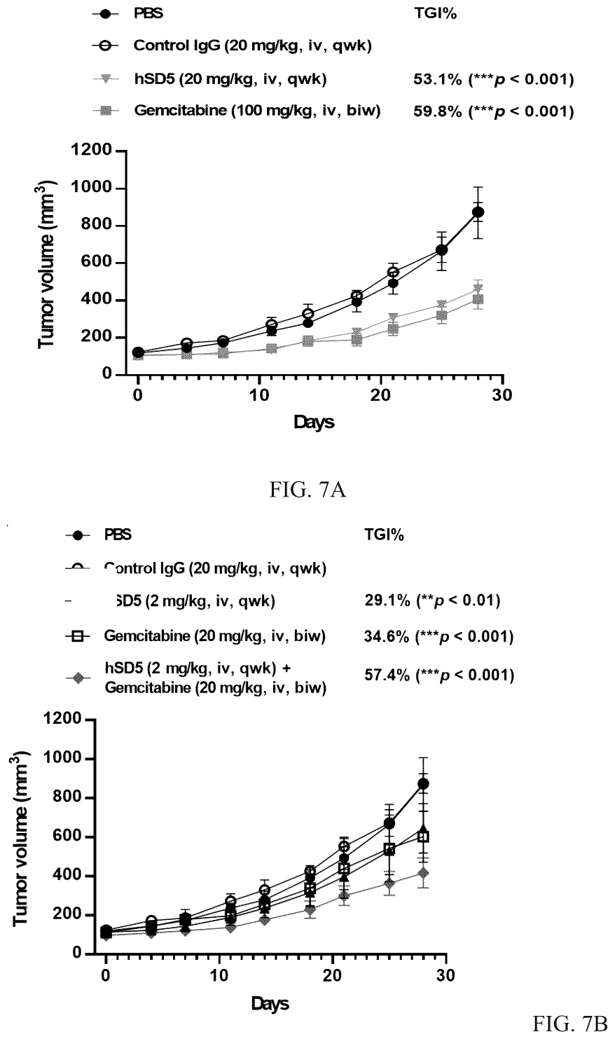

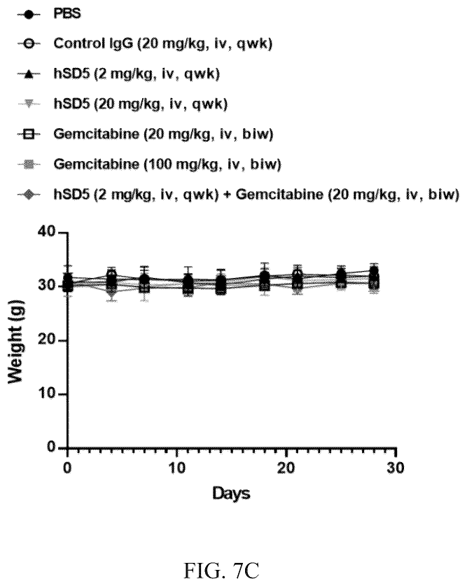

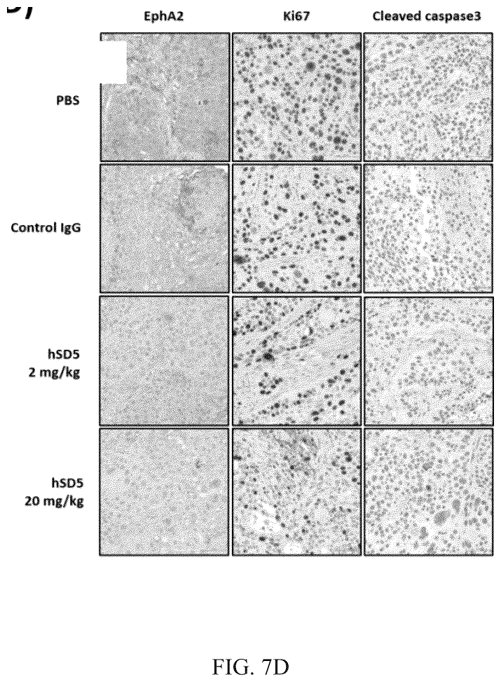

Administration of IgG hSD5 (20 mg/kg, iv, qwk, indicated as blue filled inverted triangles) and Gemcitabine (100 mg/kg, iv, biw, indicated as orange filled squares) were used to test the inhibition effect on BxPc-3 tumor growth, n=6, TGI denotes Tumor growth inhibition. (FIG. 7A) Administration of IgG hSD5 (2 mg/kg, iv, qwk, indicated by solid black triangles), Gemcitabine (20 mg/kg, iv, biw, indicated by black open squares), and the experimental group that combined IgG and Gemcitabine treatment simultaneously (indicated by solid red diamonds) were used to teat the growth inhibitory effect of BxPc-3 on the tumor. (FIG. 7B) During treatments, the weight changes of each mouse group were tracked. (FIG. 7C) The expression levels of EphA2, cell proliferation marker Ki67, and apoptosis-related marker cleaved caspase3 in the excised tumor groups were analyzed by IHC staining. (FIG. 7D)

Xenograft mice was used to evaluate the growth inhibitory effect of IgG hSD5 in vivo. In BxPc-3 xenograft mice, compared with the control IgG treatment (which exhibited no inhibitory effect), IgG hSD5-based treatment significantly inhibited the growth of tumors in vivo. The TGI of IgG hSD5 treatment of 20 mg/kg, iv, qwk was 53.1%, and that of the treatment with the Gemcitabine of 100 mg/kg, iv, qwk was 59.8% (FIG. 7A). In the same experiment, the TGI of Gemcitabine (20 mg/kg, iv, biw) was 34.6%. However, when low-dose IgG hSD5 and gemcitabine were combined, a synergistic effect was observed, with a TGI of 57.4% (FIG. 7B), and no change in the body weight of the mice was observed (FIG. 7C). Further, the IHC staining on tissue sections from the removed tumors was performed to observe the expression levels of EphA2 in BxPc-3 tumors and of the cell proliferation marker Ki-67 in the tissues. Compared with the results of control IgG group, the treatment significantly decreased the expression levels of EphA2 and Ki-67 in the tumors (FIG. 7D)

### Example 7. In Vivo Tumor Growth Inhibitory Effect of Humanized IgG hSD5 on Mia PaCa-2 Xenograft Mice

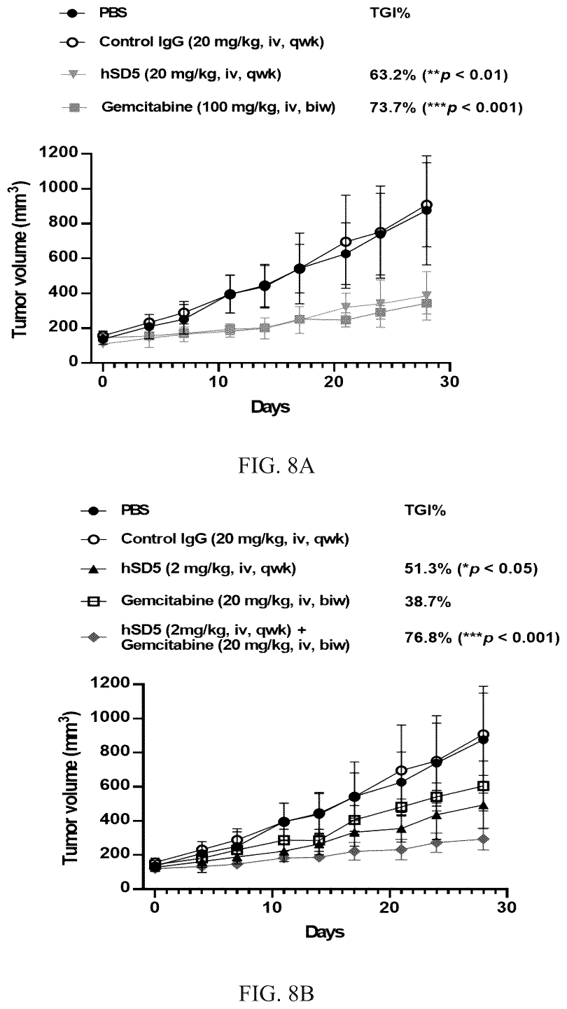

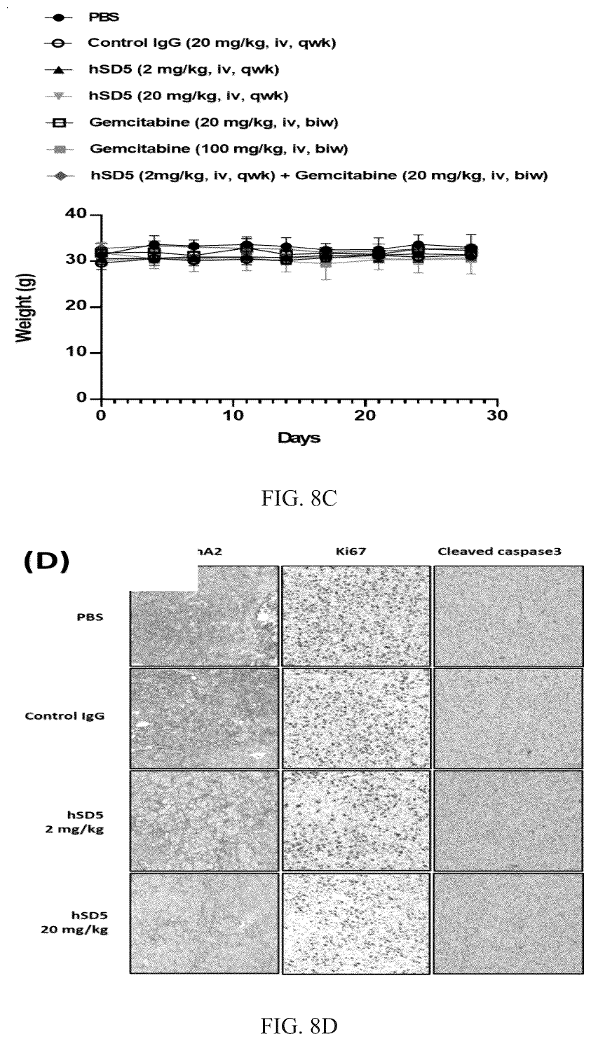

Mia PaCa-2 xenograft mice was also used to determine the inhibitory effect of IgG hSD5 on in vivo tumor growth. Administration of IgG hSD5 (20 mg/kg, iv, qwk, indicated as blue filled inverted triangles) and Gemcitabine (100 mg/kg, iv, biw, indicated as orange filled squares) were used to test the inhibition effect on BxPc-3 tumor growth, n=6, TGI denotes Tumor growth inhibition. (FIG. 8A) Administration of IgG hSD5 (2 mg/kg, iv, qwk, indicated by solid black triangles), Gemcitabine (20 mg/kg, iv, biw, indicated by black open squares), and the experimental group that combined IgG and Gemcitabine treatment simultaneously (indicated by solid red diamonds) were used to teat the growth inhibitory effect of BxPc-3 on the tumor. (FIG. 8B) During treatments, the weight changes of each mouse group were tracked. (FIG. 8C) The expression levels of EphA2, cell proliferation marker Ki67, and apoptosis-related marker cleaved caspase3 in the excised tumor groups were analyzed by IHC staining. (FIG. 8D)

After the administration of 20 mg/kg of IgG hSD5, the TGI was 63.2%, whereas after the administration of 100 mg/kg of IgG Gemcitabine, the TGI was 73.7% (FIG. 8A). When the gemcitabine (20 mg/kg) was administered, the TGIs was 38.7% (FIG. 8B). However, similarly to the therapeutic effect observed in BxPc-3 tumor-grafted mice, when the combination therapy consisting of low-dose IgG hSD5 and gemcitabine was administered, a significant synergistic effect was observed, and the TGI was 76.8%. No side effects or changes in body weight were observed in mice (FIG. 8C). Observation of the IHC staining of Mia PaCa-2 tumor tissue sections revealed that the expression levels of EphA2 and Ki-67 in the tissue significantly decreased The results demonstrated that the IgG hSD5-targeted EphA2 effectively inhibited the tumor growth in vivo (FIG. 8D).

As can be seen from Example 6 and Example 7, the administration of hSD5 (2 mg/kg, iv, qwk) in combination with gemcitabine (20 mg/kg, iv, biw) produced strong synergistic effects on the cancer cells BxPc-3 and Mia PaCa-2. These results indicate that using a low dose in combined therapy can result in tumor growth inhibition, and they also indicate the potential of antibody hSD5 for therapeutic applications. Gemcitabine is the first-line treatment drug for pancreatic adenocarcinoma; it can inhibit the synthesis of DNA after entering cells, resulting in cytotoxicity (J Clin Oncol., 15 (6): 2403-2413 (1997); Mol Pharm., 10 (2): 430-444 (2013)). However, when combined with the inhibitory effect induced by EphA2 on the surface of hSD5-targeted cancer cells, a more comprehensive therapeutic effect can be obtained in PAAD.

### Example 8. The Epitope Definition of IgG hSD5 Recognizes the Active Site of EphA2

To determine whether the antibody hSD5 can recognize two antigen fragments simultaneously, peptide synthesis of the two antigen fragments was performed to test the binding reaction of antibody IgG hSD5. The ELISA was used to test the binding reactivity of the antibody hSD5 to synthetic peptides. The two short peptides located on the designed activation site of EphA2 are EphA2pep_P1 and EphA2pep_P2, respectively. The long peptide formed by linking the two short peptides is EphA2pep_PIP2. NCIpep and NC2pep are two irrelevant peptides as the negative control. EphA2 ECD is a recombinant EphA2 extracellular domain protein.

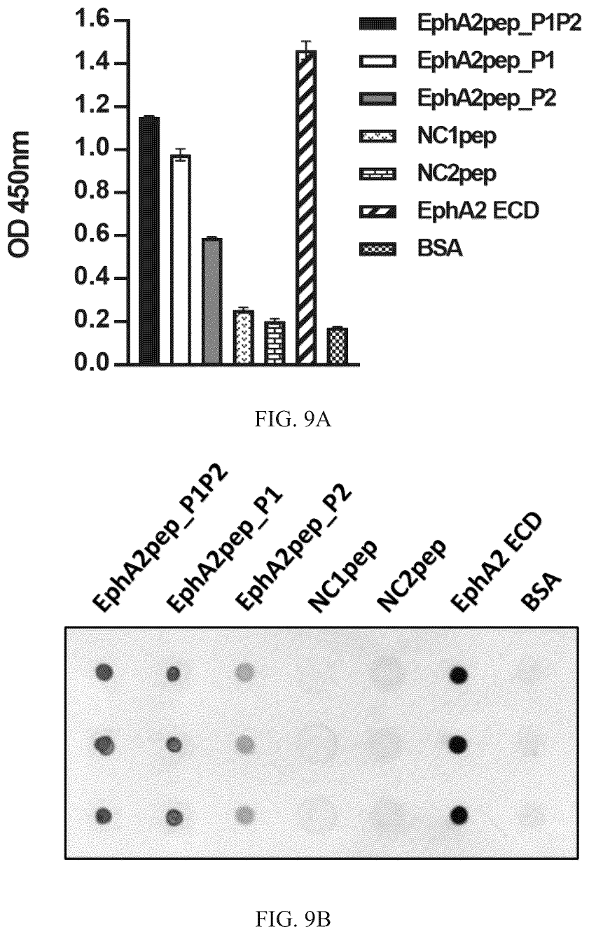

The IgG hSD5 exhibited a binding response to the long peptide (EphA2pep_PIP2) linking the two antigen fragments, and IgG hSD5 individually recognized the two synthetic short peptides (EphA2pep_P1 and EphA2pep_P2. In addition, IgG hSD5 did not exhibit a cross-binding reaction to the two irrelevant peptides. The experimental results indicate that the antibody hSD5 binds to the position of the designed antigen fragment, and that the antibody hSD5 interacts with both antigen fragments; the interaction with EphA2pep_P1 was stronger than that with EphA2pep_P2, indicating that the conformational epitope on the antibody structure can induce an immune response. (FIG. 9A)

FIG. 9B is a dot blot assay was used to test the reaction of antibody hSD5 binding to the synthetic peptide. By using the dot blotting assay, experimental findings similar to the peptide ELISA were obtained; IgG hSD5 can recognize both the antigen fragments EphA2pep_P1 and EphA2pep_P2, and their binding reaction with the peptide EphA2pep_P1 was stronger than that with EphA2pep_P2. (FIG. 9B)

In the therapeutic strategy targeting EphA2, the use of soluble ephrin A1 or fusing recombinant ephrin A1 to human IgG Fc for dimerization can effectively promote the phosphorylation and degradation of EphA2 and ultimately inhibit the growth of tumor cells (Biochem Biophys Res Commun. 320 (4): 1096-1102 (2004)). However, ephrin A1 interacts with multiple Eph family molecules, and these factors may produce adverse side effects, limiting its efficacy. Through structural design for immunogens, we prepared specific antibodies targeting the binding of ephrin A1 at the activation site on EphA2; the experimental results indicate that the antibody can bind to the conformational epitope formed by two discontinuous surfaces (FIGS. 9A and 9B). Therefore, the antibody hSD5 can induce a forward tumor growth inhibitory effect similar to that of ephrin A1 targeting EphA2. As demonstrated earlier in FIG. 6A of Example 5, the hSD5 binds specifically to EphA2 and does not cross-bind to other Eph family proteins. This result reflects the advantages of antibodies: preventing adverse side effects and facilitating inhibitory reactions that neutralize the EphA2 molecule.

### Example 9. The Growth Inhibitory Response of Different Pancreatic Cancer Cell Lines Treated with Serially Diluted MMAE

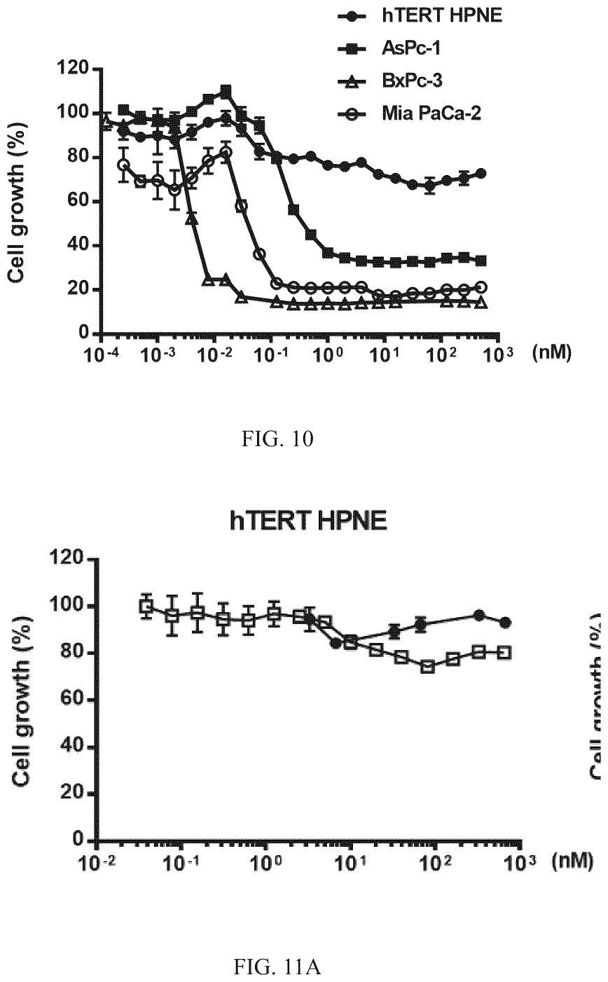

As shown in FIG. 10, the hTERT-HPNE is a normal pancreatic endothelial cell line.

Monomethyl auristatin E or MMAE is 100-1000 times more potent than doxorubicin (Adriamycin/Rubex) and cannot be used as a drug itself. However, as part of an antibody-drug conjugate or ADC, MMAE is linked to a monoclonal antibody (mAb) that recognizes a specific marker expression in cancer cells and directs MMAE to a specific, targeted cancer cell. (Int J Mol Sci. 21 (9): 3286 (2020)) In this example, the efficacy of MMAE on the pancreatic cancer cell line, AsPc-1, BxPc-3, Mia PaCa-2. On the contrary, it exhibited only mild effect on the growth inhibition of normal pancreatic cell line, hTERT HPNE. (FIG. 10)

### Example 10. The Growth Inhibitory Response of Different Pancreatic Cancer Cell Lines by Administration of Serially Diluted hSD5-ADC

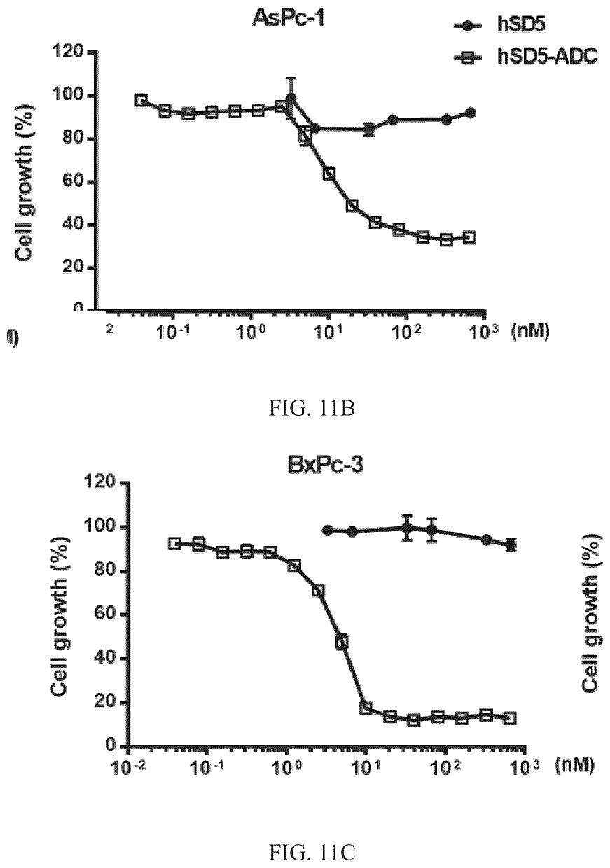

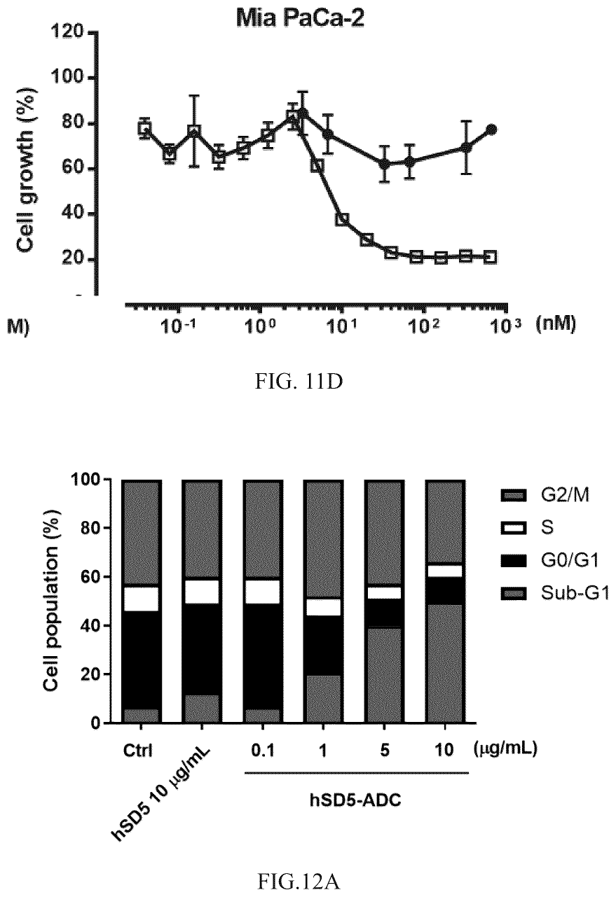

As can be seen from FIGS. 11A-11D, it is apparent that administering hSDS alone does not provide ideal inhibition effect on various pancreatic cell lines. (hTERT HPNE, AsPC-1, BxPc-3, and Mia PaCa-2), whereas administering hSD5-ADC provides significant effect on the growth inhibitory response of those pancreatic cell lines. Since the hSD5-ADC has limited effect on the normal pancreatic cell line, it can be concluded that hSD5-ADC have displayed high selectivity towards the pancreatic cancer cell lines (AsPC-1, BxPc-3, and Mia PaCa-2).

### Example 11. The Cell Cycle Changes of Pancreatic Cancer Cells

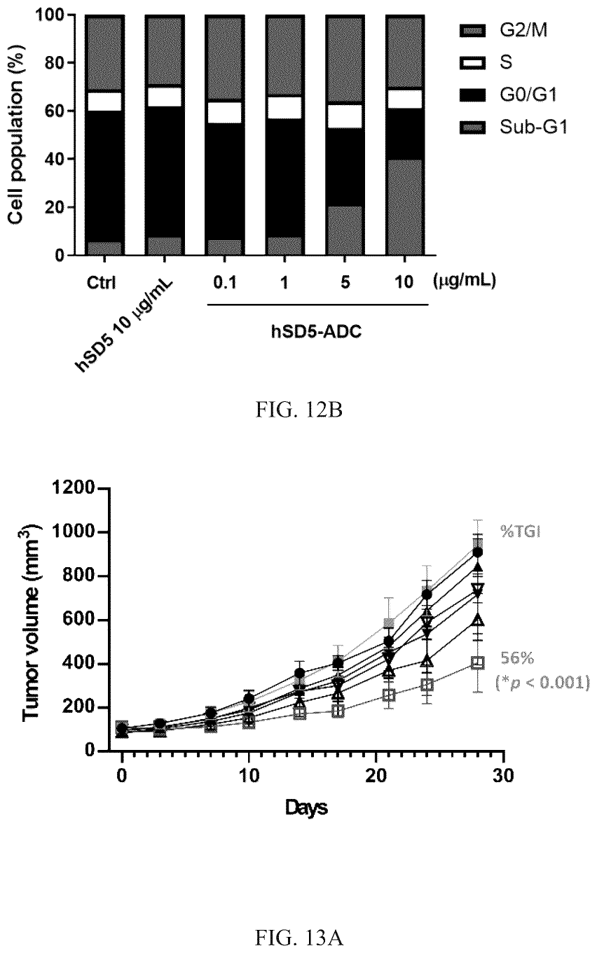

FIGS. 12A-12B illustrates the percentage of cells population in different phases of the cell cycle in the stacked bar graphs. The hSD5 is the same antibody without MMAE as a comparison group. Higher population of the apoptotic cells (indicated by the Sub-G1 bar) in both the BxPc-3 (FIG. 12A) and Mia PaCa-2 (FIG. 12B) cell lines after treatment with different doses of hSD5-ADC suggest that hSD5-ADC is effective on inducing cancer cell death. A dose dependency of the hSD5-ADC response is also observed.

### Example 12. The BxPc-3 Xenograft Mouse Model was Used to Test the Inhibitory Effect on Tumor Growth in Mice after Administration of Different Concentrations of hSD5-ADC and Control Group IgG-ADC

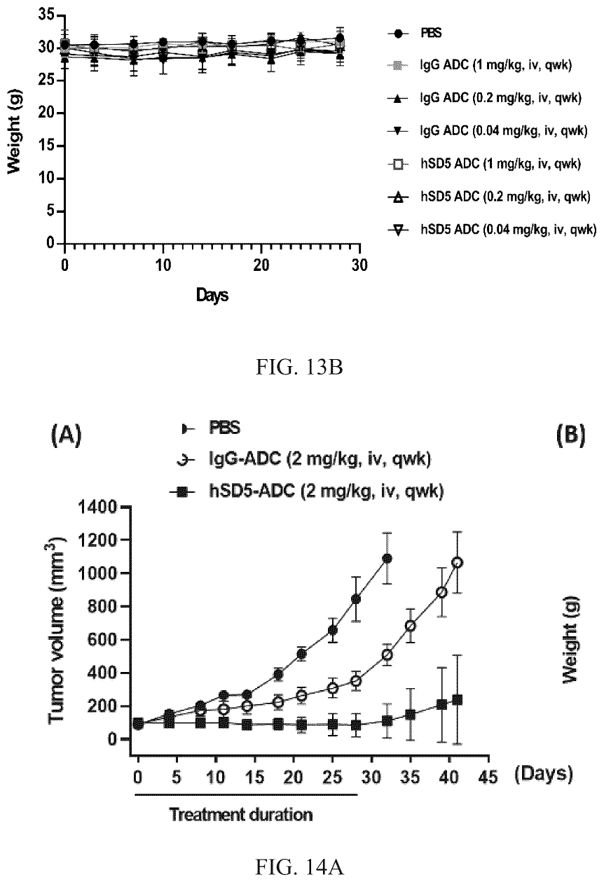

The tumor size was measured during the administration of antibody therapy. The conditions of antibody administration were tail vein i.v. injection once a week, n=5. The % TGI was the percentage of tumor growth inhibition. (FIG. 13A) The body weight changes of mice in each group were recorded during antibody treatment. (FIG. 13B)

As can be seen from FIG. 13A, the inhibitory effect on tumor growth upon administration of IgG-ADC is limited, whereas the inhibitory effect on tumor growth upon administration of hSD5-ADC is more significant. (The TGI of hSD5-ADC treatment of 1 mg/kg, iv, qwk was 56%). A dose dependent trend is also observed.

As can be seen from FIG. 13B, it is apparent that no side effects or changes in body weight were observed in mice.

### Example 13. In Vivo Tumor Growth Inhibitory Effect of hSD5-ADC on BxPc-3 Xenograft Mice

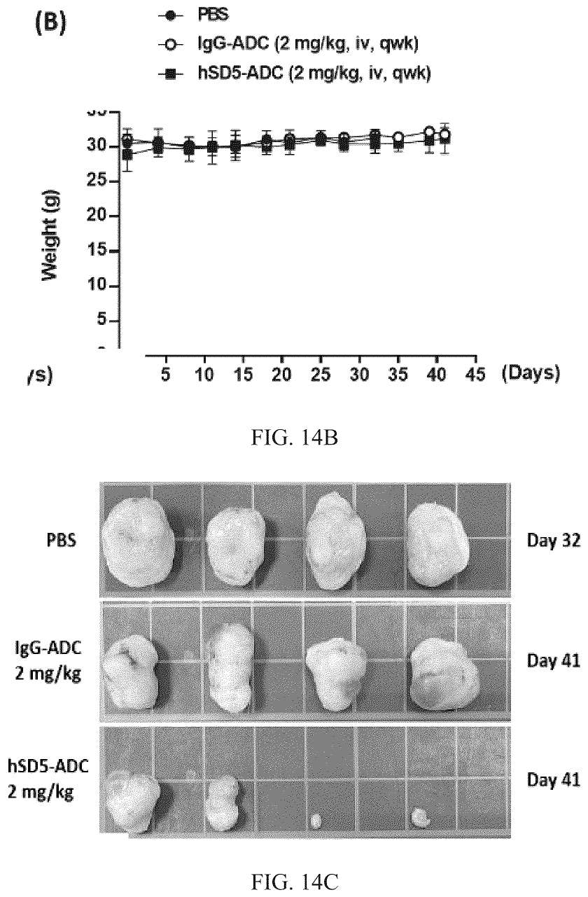

The hSD5-ADC was administered to the mice (2 mg/kg, iv, qwk) to test the tumor growth inhibition effect. The IgG-ADC was used as the experiment's control group under the same administration conditions, n=4. (FIG. 14A) During treatments, the weight changes of each mouse group were tracked. (FIG. 14B) After the experiment was completed, the tumor in the mouse was taken out for recording. (FIG. 14C)

As can be seen from FIGS. 14A and 14C, there is only mild inhibitory effect on tumor growth with IgG-ADC administration, whereas the inhibitory effect on tumor growth is significant with hSD5-ADC administration. As can be seen from FIGS. 14B, it is apparent that no side effects or changes in body weight were observed in mice.

### Example 14. Anti-EphA2 hSD5 could Recognize the Endogenous EphA2 Molecule and Inhibit the Cell Growth on Gastric Cancer

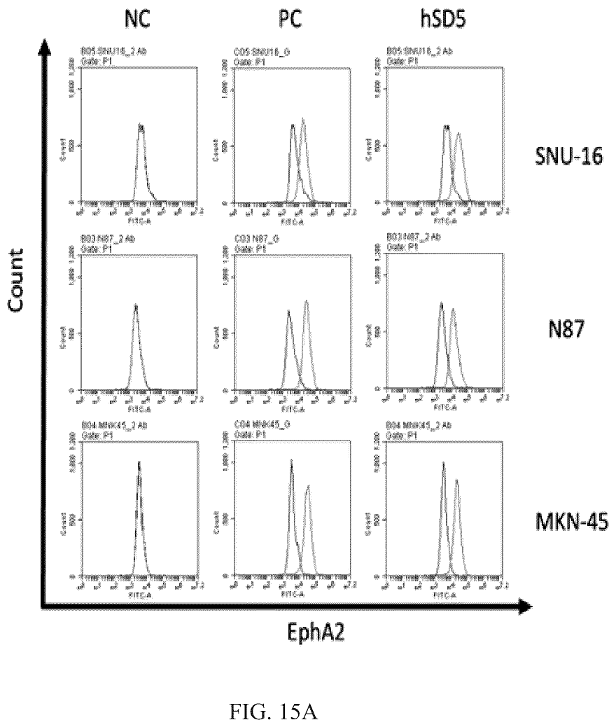

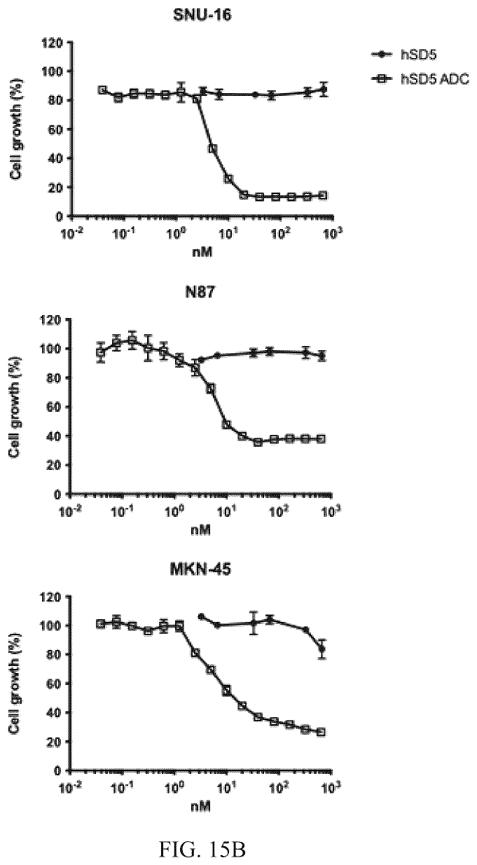

Flow cytometry was used to analyze the response of hSD5 IgG in identifying endogenous EphA2 molecules on the three strains of gastric cancer cell lines. PC represents the use of commercial Ab to determine the expression of EphA2 on cancer cells; NC represents the control group with irreverent of the isotype IgG1 antibody. The hSD5 was used to identify endogenous EphA2 molecules on three strains of gastric cancer cells by flow cytometry analysis. The results indicate that the hSD5 exhibited a significant binding response to the EphA2 molecules on gastric cancer cells. (FIG. 15A)

The growth inhibitory effect of hSD5-ADC on the three strains of gastric cancer cells were tested under different concentrations. At indicated concentrations interacted with the cancer cells for 3 days, and the effects on the growth of cancer cells were observed by MTS assay. As can be seen from FIG. 15B, it is apparent that administering hSDS alone does not provide ideal inhibition effect on three different gastric cancer cells (SNU-16, N87 and MKN-45), whereas administering hSD5-ADC provides significant effect on the growth inhibitory response of those gastric cancer cell lines. The result suggests that t hSD5-ADC have displayed high selectivity towards the gastric cancer cell cell lines. Besides, a dose-dependent inhibition of hSD5 and hSD5 ADC was observed, and the growth inhibitory effect of hSD5-ADC on the three strains of gastric cancer cells were more significant that thaf of hSD5. (FIG. 15B)

### Example 15. The Expression of EphA2 and the Binding Ability of Anti-EphA2 hSD5 on GBM

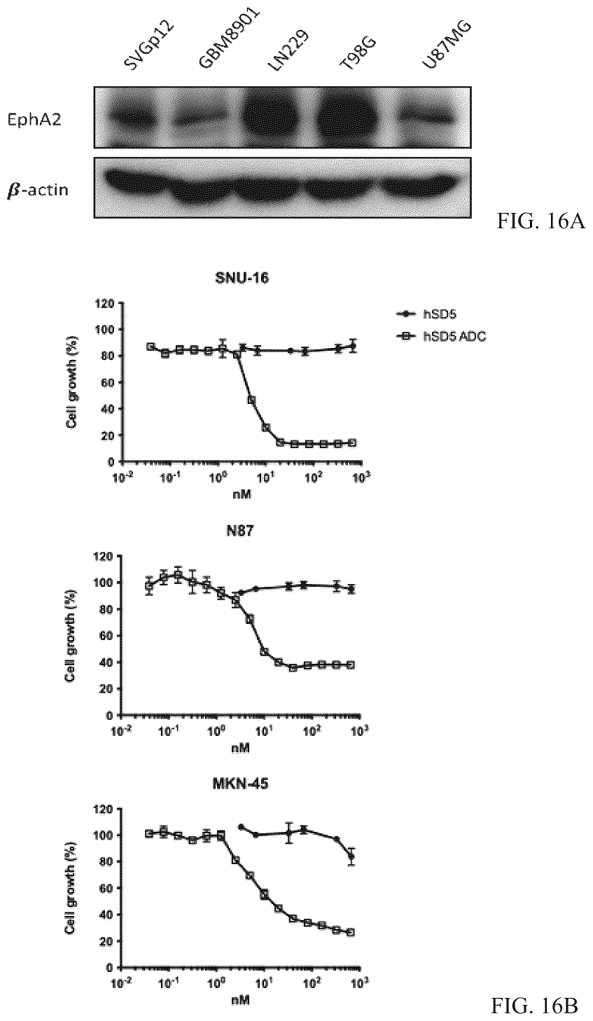

To analyze the expression of EphA2 molecules, four brain tumor cell lines, GBM8901, LN229, T98G and U87MG, and one normal cell line SVGp12 were used to compare the expression of EphA2 in cancer cells. All four brain tumor cell lines exhibited moderate or higher expression of the EphA2 protein compared to that of the normal SVGp12 cell line (FIG. 16A)

Flow cytometry was used to analyze the response of hSD5 IgG in identifying endogenous EphA2 molecules on the four strains of brain tumor cell lines. PC represents the use of commercial Ab to determine the expression of EphA2 on cancer cells; NC represents the control group with irreverent of the isotype IgG1 antibody. (FIG. 16B)

The hSD5 was used to identify endogenous EphA2 molecules on the four brain tumor cell lines by flow cytometry analysis. PC uses a commercial anti-EphA2 antibody, and NC is the control group with irreverent of the isotype IgG1. The results indicate that the hSD5 exhibited a significant binding response to the EphA2 molecules on brain cancer cells.

### Example 16. Anti-EphA2 hSD5 could Recognize the Endogenous EphA2 Molecule and Inhibit the Cell Growth on Cholagiocarcinoma and Bladder Cancer

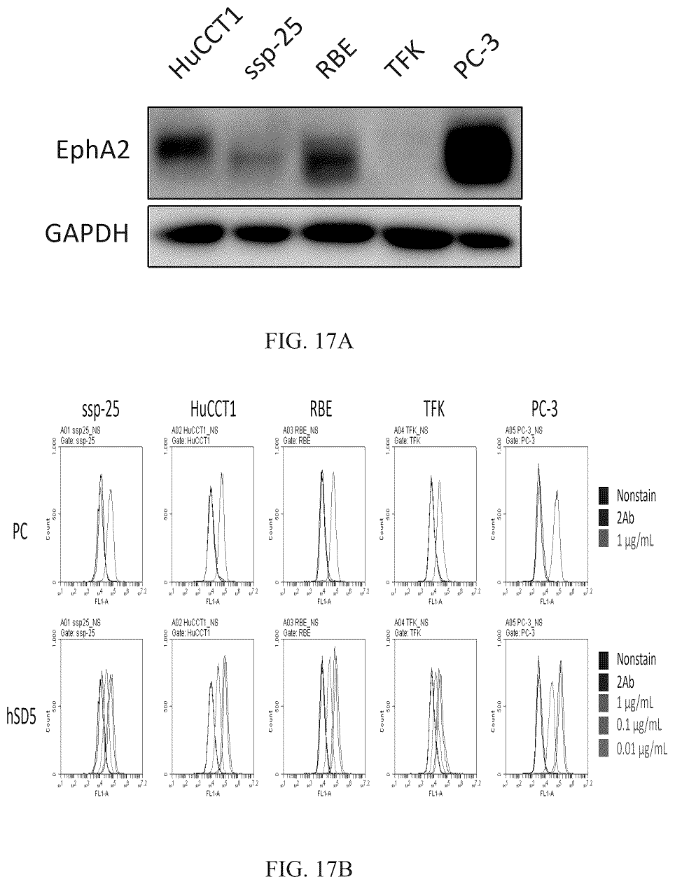

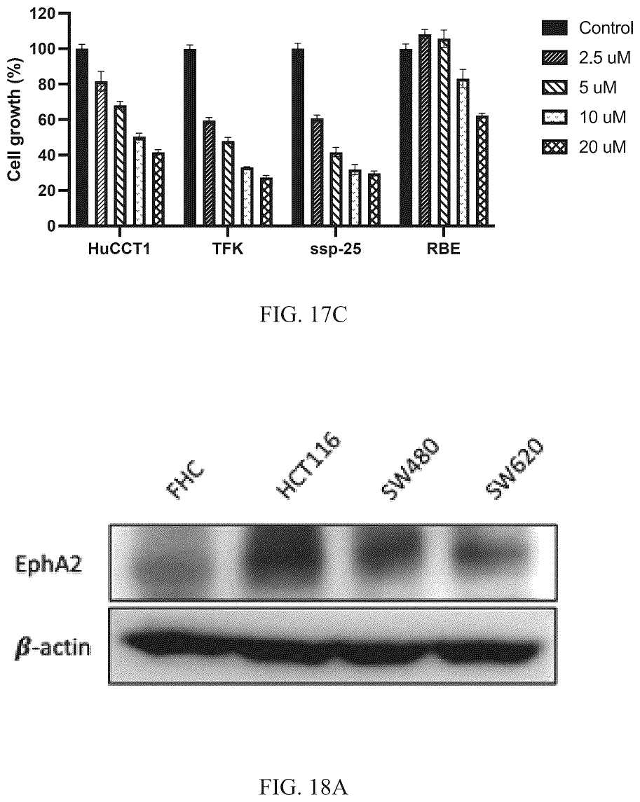

The expression of EphA2 molecules in four cholagiocarcinoma cell lines, HuCCT1, ssp-25, RBE, TFK, and one bladder cancer cell, PC-3, were analyzed using Western blotting. (FIG. 17A) Four cholagiocarcinoma and one bladder cancer cell lines exhibited moderate to high expression of the EphA2 protein than did the control cell line. The results of the expression of EphA2 molecules correlates with the binding response to the EphA2 molecules in the four cholagiocarcinoma cell lines.

Flow cytometry was used to analyze the response of hSD5 IgG with different concentrations in identifying endogenous EphA2 molecules on the four strains of cholagiocarcinoma cell lines and bladder cancer cell line. PC represents the use of commercial Ab to determine the expression of EphA2 on cancer cells. (FIG. 17B) The results indicate that the hSD5 exhibited a significant binding response to the EphA2 molecules on cancer cells.

The growth inhibitory effect of hSD5 scFv on the four strains of cholagiocarcinoma cells was tested under different concentrations. At indicated concentrations, hSD5 scFv interacted with the cancer cells for 5 days, and the effects on the growth of cancer cells were observed by MTS assay. (FIG. 17C)

By Day 5 of the antibody reaction, at a concentration of 20 mM, hSD5 scFv inhibited the growth of the four cell lines HuCCT1, ssp-25, RBE, abd TFK by approximately 40%-70% inhibitory effect on the cancer cell lines %, respectively. Dose-dependent responses were observed at different antibody concentrations; this result indicates that the binding of hSD5 scFv to EphA2 on the surface of cholagiocarcinoma cell can inhibit the growth of cancer cells.

### Example 17. Anti-EphA2 hSD5 could Recognize the Endogenous EphA2 Molecule and Inhibit the Cell Growth on Colon Cancer

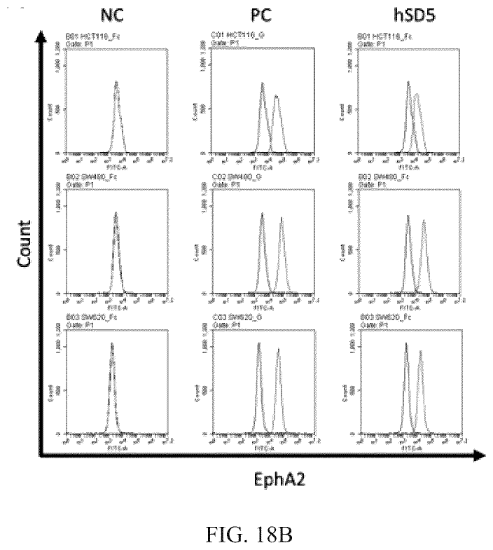

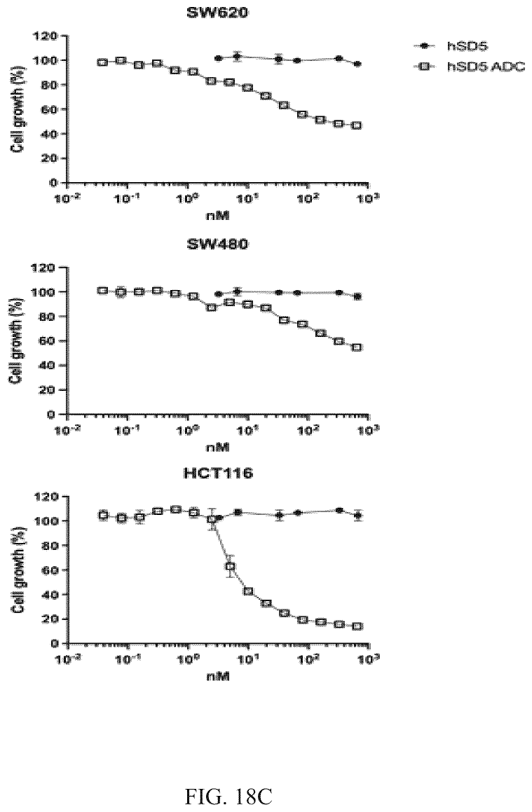

The expression of EphA2 molecules in three colon cancer cells, HCT116, SW480 and SW460, and one normal colon endothelial cell, FHC, were analyzed using Western blotting. (FIG. 18A) All three colon cancer cell lines exhibited higher expression of the EphA2 protein than that of the control cell line.

Flow cytometry was used to analyze the response of hSD5 IgG in identifying endogenous EphA2 molecules on the three strains of gastric cancer cell lines. PC represents the use of commercial Ab to determine the expression of EphA2 on cancer cells; NC represents the control group with irreverent of the isotype IgG1 antibody. (FIG. 18B) The results indicate that the hSD5 exhibited a significant binding response to the EphA2 molecules on cancer cells.

The growth inhibitory effect of hSD5-ADC on the three strains of colon cancer cells were tested under different concentrations. At indicated concentrations interacted with the cancer cells for 3 days, and the effects on the growth of cancer cells were observed by MTS assay. (FIG. 18C)

As can be seen from FIG. 18C, it is apparent that administering hSDS alone does not provide ideal inhibition effect on three different colon cancer cells (HCT116, SW480 and SW460), whereas administering hSD5-ADC provides significant effect on the growth inhibitory response of those colon cancer cell lines. The result suggests that t hSD5-ADC have displayed high selectivity towards the colon cancer cell cell lines. Besides, a dose-dependent inhibition of hSD5 and hSD5 ADC was observed, and the growth inhibitory effect of hSD5-ADC on the three strains of colon cancer cells were more significant that thaf of hSD5.

### Example 18. The hSD5-ADC can Inhibit Tumor Growth on HCT116 Xenograft Mouse Model

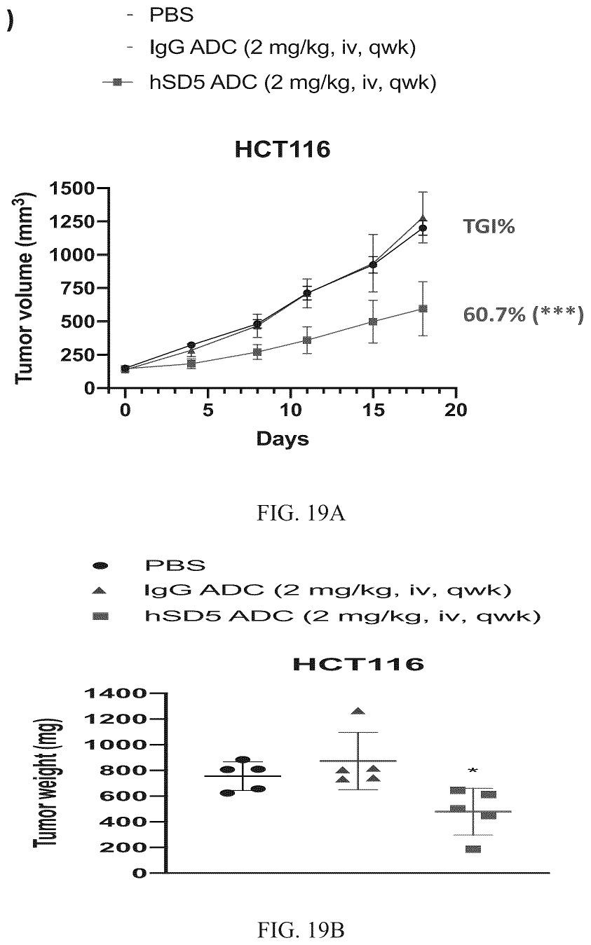

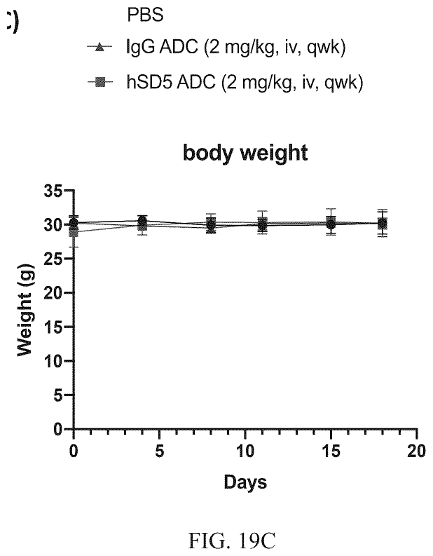

The growth inhibitory effect of hSD5-ADC (2 mg/kg, iv, qwk, labeled with a solid red square), control IgG-ADC (2 mg/kg, iv, qwk, labeled with a solid blue triangle) and PBS group (labeled with a solid black circle) on HCT116 tumor volume were observed. (FIG. 19A) The tumor weight of HCT116 xenograft tumor after hSD5-ADC and control IgG-ADC treatment (* p<0.05.) (FIG. 19B) After administering antibodies, the weight changes in the HCT116 xenograft mice were recorded. (FIG. 19C). As can be seen from FIGS. 19A-19B, the inhibitory effect on tumor growth upon administration of IgG-ADC is limited, whereas the inhibitory effect on tumor growth upon administration of hSDS-ADC is more significant. (The TGI of hSD5-ADC treatment of 2 mg/kg, iv, qwk was 60.7%). As can be seen from FIG. 19C, it is apparent that no side effects or changes in body weight were observed in mice.

What is claimed is:

1. An isolated anti-EphA2 antibody or an antigen-binding portion thereof, comprising a light chain CDR1 (L-CDR1) comprising the amino acid residue of SEQ ID NO: 1, or a variant having an amino acid sequence with at least 95% identity to SEQ ID NO: 1; a light chain CDR2 (L-CDR2) comprising the amino acid residue of DND, or a variant having an amino acid sequence with at least 95% identity to DND; and a light chain CDR3 (L-CDR3) comprising the amino acid residue SEQ ID NO: 3, or a variant having an amino acid sequence with at least 95% identity to SEQ ID NO: 3; and
a heavy chain complementarity determining region 1 (H-CDR1) comprising the amino acid residue of SEQ ID NO: 4, or a variant having an amino acid sequence with at least 95% identity to SEQ ID NO: 4; a heavy chain CDR2 (H-CDR2) comprising the amino acid residue of SEQ ID NO: 5, or a variant having an amino acid sequence with at least 95% identity to SEQ ID NO: 5; and a heavy chain CDR3 (H-CDR3) comprising the amino acid residue of SEQ ID NO: 6, or a variant having an amino acid sequence with at least 95% identity to SEQ ID NO: 6.

2. The anti-EphA2 antibody or the antigen-binding portion thereof of claim 1, which is a monoclonal antibody, chimeric antibody, humanized antibody or human antibody.

3. The anti-EphA2 antibody or the antigen-binding portion thereof of claim 1, which is a single chain Fv (scFv), IgG, Fab, (Fab)₂, or (scFv′)₂.

4. The anti-EphA2 antibody or the antigen-binding portion thereof of claim 1, comprising
a light chain comprising an amino acid sequence comprising SEQ ID NO: 7 or 8, or a variant having at least 95% identity to SEQ ID NO: 7 or 8; and
a heavy chain comprising an amino acid sequence comprising SEQ ID NO: 9 or 10, or a variant having at least 95% identity to SEQ ID NO: 9 or 10.

5. The anti-EphA2 antibody or the antigen-binding portion thereof of claim 4, comprising
a light chain comprising the amino acid sequence of SEQ ID NO: 7 or 8; and
a heavy chain comprising the amino acid sequences of SEQ ID NO: 9 or 10.

6. The anti-EphA2 antibody or the antigen-binding portion thereof of claim 1, comprising the amino acid sequence of SEQ ID NO: 11 or 12, or a variant having at least 95% identity to SEQ ID NO: 11 or 12.

7. The anti-EphA2 antibody or the antigen-binding portion thereof of claim 6, comprising the amino acid sequence of SEQ ID NO: 11 or 12.

8. The anti-EphA2 antibody or the antigen-binding portion thereof of claim 1, wherein the antibody is a humanized antibody.

9. An antibody-drug conjugate (ADC), comprising the anti-EphA2 antibody or an antigen-binding portion thereof of claim 1 and a drug-linker structure comprising an antitumor compound connected to the antibody by a linker.

10. The antibody-drug conjugate of claim 9, wherein the antitumor compound is selected from auristatins such as monomethyl auristatin E (MMAE) and monomethyl auristatin F (MMAF), vincristine, vinblastine, methotrexate, platinum-based antitumor agents (cisplatin and derivatives thereof), doxorubicin, calicheamicin, dolastatin 10, maytansinoids, a pyrrolobenzodiazepine dimer, a camptothecin derivative, duocarmycins, amanitin, daunorubicin, mitomycin C, bleomycin, cyclocytidine, and Taxol and derivatives thereof.

11. The antibody-drug conjugate of claim 9, wherein the antitumor compound is MMAE.

12. A pharmaceutical composition comprising the ADC of claim 9 and a pharmaceutically acceptable carrier or excipient.

13. The pharmaceutical composition of claim 12, which further comprises or is used in combination with one or more additional anticancer agents.

14. The pharmaceutical composition of claim 13, wherein the one or more additional anticancer agents is Gemcitabine.

15. A method for treating or preventing a EphA2 associated cancer in a subject, comprising administering a therapeutically effective amount of the ADC of claim 9 to the subject.

16. A method for inhibiting EphA2 associated cancer cell growth or cancer metastasis in a subject comprising administering a therapeutically effective amount of the ADC of claim 9 to the subject.

17. The method of claim 15, wherein the EphA2 associated cancer is selected from bile duct cancer, bladder cancer, brain cancer, breast cancer, cervical cancer, colon cancer, esophageal cancer, gastric cancer, gliomas, liver cancer, lung cancer, melanoma, ovarian cancer, pancreatic cancer, prostate cancer, rectal cancer, renal cancer, stomach cancer, thymus cancer, and vulvar cancer.

18. (canceled)

19. The method of claim 15, which further comprises administering an additional anti-cancer agent.

20. The method of claim 19, wherein the additional anti-cancer agent is gemcitabine.

21. A kit for detecting or diagnosing a EphA2 associated cancer or an elevated risk of future occurrence of a EphA2 associated cancer, or predicting a metastasis or prognosis of a cancer, or monitoring cancer progression in a subject, comprising the anti-EphA2 antibody or the antigen-binding portion thereof of claim 1.
